(19) United States
(12) Patent Application Publication
(10) Pub. No.: US 2024/0207491 A1
(43) Pub. Date: Jun. 27, 2024

(54) Development and Vascular Applications of Shape Memory External Stents

(71) Applicants: VANDERBILT UNIVERSITY, Nashville, TN (US); THE UNITED STATES GOVERNMENT AS REPRESENTED BY THE DEPARTMENT OF VETERANS AFFAIRS, Washington, DC (US)

(72) Inventors: Timothy C. Boire, Nashville, TN (US); Hak-Joon Sung, Nashville, TN (US); Colleen Brophy, Nashville, TN (US)

(21) Appl. No.: 18/380,135

(22) Filed: Oct. 13, 2023

Related U.S. Application Data
(63) Continuation of application No. 15/567,033, filed on Oct. 16, 2017, now abandoned.
(60) Provisional application No. 62/148,164, filed on Apr. 15, 2015.

(51) Int. Cl. A61L 31/06; A61F 2/07; A61F 2/90; A61L 31/04; A61L 31/16; C08G 63/08

(52) U.S. Cl. CPC A61L 31/06; A61F 2/07; A61F 2/90; A61L 31/041; A61L 31/16; C08G 63/08; A61L 2400/16

(57) ABSTRACT

The presently-disclosed subject matter includes a compound comprising a first monomer, which is allyl-functionalized and crosslinkable, and a second monomer, which is not crosslinkable. In some embodiments the compounds are photocrosslinkable, and in certain embodiments are photo crosslinkable by ultraviolet light. Also provided are shape memory vascular grafts comprised the of present compounds that can transition from a temporary shape to an original shape when heated above a melting temperature of the graft. Still further provided are methods for treating vascular conditions that utilize embodiments of the present grafts.

### CROSS-REFERENCE TO RELATED APPLICATIONS

This application claims benefit of the following patent application(s) which is/are hereby incorporated by reference: Ser. No. 15/567,033 filed on Oct. 16, 2017, which was a 371 application of International Patent Application Number PCT/US2016/027901 filed Apr. 15, 2016 that further claimed priority from provisional application No. 62/148,164 filed Apr. 15, 2015.

### GOVERNMENT INTEREST

This invention was made with government support under Grant Number CBET 1219573 awarded by the National Science Foundation. The government has certain rights in the invention.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### TECHNICAL FIELD

The presently-disclosed subject matter relates to shape memory polymers. In particular, the presently-dis-closed subject matter relates to vascular grafts comprised of allyl-functionalized shape memory polymers as well as methods of treating vascular conditions using the same.

### INTRODUCTION

The present invention relates generally to Vascular conditions can often lead to severe complications or even death. Such vascular conditions include, but are not limited to, hemorrhages, aneurysms, occlusions: and ischemic tissue. Vascular conditions also present unique treatment challenges. This is particularly so when treating vessels that are small or difficult to access. For instance, traditional surgical treatment techniques are invasive to surrounding tissue and can be costly, can result in a high amount of pain, and can require a lengthy recovery.

In this regard, this regard, thermo-responsive shape memory polymers (SMPs) have drawn extensive interest in a wide range of applications, including biomedical, aero-space, self-healing, and textile applications. See, for example, Xue et al. Synthesis and characterization of elastic star shaped-memory polymers as self-expandable drug-eluting stents. J Material Chemistry 2012: 22(15). Such SMPs can recover their original shape after being programmed into a distinct temporary shape. Poly(ε-caprolactone) (PCL) is an exemplary biocompatible, biodegradable polymer FDA-approved for specific biomedical applications that can be chemically modified and cross-linked to form SMPs. How-ever, its melting temperature (Tₘ) of 45° C. to 60° C. is too high for physiological applications (37° C.). Thus, SMPs such as PCL have limited clinical capabilities in the treatment of vascular and other conditions. Furthermore, the use of other SMPs for therapeutic purposes has been hampered. They require an additional methacrylate functionalization step or a multistep monomer synthesis scheme.

Hence, there remains a need for compositions and methods for treating vascular conditions that are relatively noninvasive, painless, and inexpensive. There also remains a need for SMPs that can be used for such applications and that have melting points that are suited for physiological applications.

One embodiment of the present invention is a mechanically compliant, moldable, shape memory external support that can be custom fit around a vascular graft anastomosis to prevent neointimal formation. This embodiment can also provide localized, sustained delivery of therapeutics with anti-neointimal effects.

Other embodiments include nastomotic stents using a composition disclosed herein, including a novel class of poly(ε-caprolactone) (PCL)-based shape memory polymers (SMPs), PCL-co-(α-allyl carboxylate ε-caprolactone) (x% PCL-y% ACPCL)[x% and y%: molar percentages], and designed to address the design criteria developed for external stenting of vascular and hemodialysis grafts. An anti-neointimal therapeutic can also be incorporated in the matrix for localized, sustained release and potentially have a more pronounced therapeutic effect.

External mesh supports applied to vein grafts have demonstrated promise to inhibit intimal hyperplasia by promoting “outward remodeling” (arterialization) accompanied by adventitial microvessel growth (i.e., neo-vasa vasorum formation). The materials used to date, however, are highly rigid and inflexible, in contrast to ‘the compliant nature of the artery. This precludes application to the anastomoses, where failure commonly occurs, especially in hemodialysis access patients that receive PTFE access grafts. Rigidity also increases restenotic risks and makes it difficult to control spacing between the external support and vein. Previous studies have shown that these meshes should be fitted loosely around the grafts, and asymmetric wrapping creates non-uniform neo-vasa vasorum formation turbulent flow, and neointimal formation, especially around the anastomotic sites. The present invention overcomes this issue by developing a new class of shape memory external support that enables custom fitting to each vein anastomosis to promote more uniform outward instead of inward remodeling and locally delivering anti-neointimal therapeutics over time.

Embodiments of the present invention are biocompatible, biodegradable SMPs, and can be custom fit to anastomoses to promote uniform vein-to-stent spacing and outward remodeling beneficial towards neointimal abrogation.

Embodiments of the present invention incorporate novel SMPs that maintain healthy vascular cell phenotypes with regulated redox potential for improved vein patency.

Embodiments of the present invention comprise SMPs that are mechanically compliant to enable vein contractility and provide arterymimetic mechanical support in the arterial circulation, thereby mitigating neointimal formation arising from compliance mismatch and arterial hemodynamic effects.

Embodiments of the present invention incorporate SMPs that degrade slowly, enabling sustained mechanical support during pivotal venous adaptation.

Embodiments of the present invention incorporate SMPs that are easily deployed over the PTFE graft onto the venous anastomosis and, as such, can provide site-directed therapeutic intervention.

Adventitial application of therapeutic materials and drugs allows for more efficient minimization of intimal hyperplasia by enabling closer contact with the myofibro-blasts/vascular smooth muscle cells (VSMCs) and maintaining higher drug concentrations with fewer toxicity concerns.

Embodiments of the present invention may incorporate an anti-neointimal therapeutic can be incorporated into the matrix (see Table 1 below).

TABLE 1

| Anti-neointimal | | |
|---|---|---|
| agents | Molecular target | Therapeutic action |
| Anti-neointimal | MK2 | Fibrosis, inflammation, |
| peptide | | migration, proliferation |
| Rapamycin | mTOR | Proliferation |
| Tacrolimus | FKBPs | Inflammation |
| Marimastat | MMPs | Migration |
| Dexamethasone | GR | Inflammation, migration, |
| | | proliferation |
| Pioglitazone | PPARy | Proliferation |
| AZX | HSP20 | Migration, fibrosis |
| Cilistazol | Adenylate | Migration, Inflammation |
| | cyclase | |

Other embodiments are described herein, specifically including ATTACHMENTS 1-4. α

### BRIEF DESCRIPTION OF THE DRAWINGS

FIGS. 1A to 1E include (FIG. 1A) a synthetic scheme of α-allyl carboxylate ε-caprolactone (ACCL), (FIG. 1B) 1H-NMR spectrum of ACCL, (FIG. 1C) a syn-thetic scheme for an x% PCL-y% ACPCL SMP network, (FIG. 1D) 1H-NMR spectrum of a 96% PCL-04% ACPCL copolymer, and (FIG. 1E) a graph of ACCL:CL feed ratio versus actual x% PCL-y% ACPCL molar composition.

FIGS. 2A and 2B include (FIG. 2A) a synthetic scheme for 100% PCL-dimethacrylate control, and (FIG. 2B) 1H-NMR spectra of 100% PCL (top) and 100% PCL-dimethacrylate (bottom).

FIG. 3 includes a graph showing the correlation between y% ACPCL and thermal properties of crosslinked SMP networks.

FIGS. 4A to 4C include stress-controlled thermomechanical cycling of (FIG. 4A) crosslinked 96% PCL-4% ACPCL, (FIG. 4A) crosslinked 89% PCL-11% ACPCL, and (FIG. 4C) 100% PCL-dimethacrylate SMP networks, where SMP films were (1) heated above their Tₘ and programmed into an elongated shape by subjecting to tensile stress (0.004 MPa min⁻¹ to 0.039 MPa), (2) cooled (2° C. min⁻¹ to 0° C.) to yield the maximum strain, ε₁(N), (3) relieved of stress (0.004 MPa min⁻¹ to O MPa) to yield the temporary shape, ε_{J}(N), (4) heated (2° C. min¹) above Tₘ yielded the original shape, εₚ(N).

FIGS. 5A to 5F include shape memory demonstrations for 88% PCL-12% ACPCL showing a (FIG. 5A) tubular original shape that is (FIG. 5B) deformed into a thread by heating at 50° C. applying strain, and fixing in an ice bath, (FIG. 5C) heating at 37° C. to recover the original tube shape, as well as (FIG. 5D) 94% PCL-06% ACPCL guitar shape (FIG. 5E) heated to 50° C. strained, contorted, and fixed at 4° C. before (FIG. 5F) ultimate recovery of the original guitar shape at 48° C.

FIG. 6 includes a chart showing the covariance between physicochemical and thermal, mechanical, and shape memory properties for a photocrosslinked SMP library, wherein the degree of covariance between properties is represented by the color and annotated values, indicating the nature of correlation between the variables (y%=y% ACPCL; X_{g}=X_{G}; Mₙ=Mₙ; MW=M_{w}; Tm=Tₘ; Hm=ΔHₘ; Tc=T_{c}; Etn=E′(37° C.); Snmax=εₘₐₓ; Ssmax=σmax; Rr=Rᵣ (N); Rf=R_{f}/N)).

FIG. 7 includes a graph showing the viability of HUVECs seeded directly on polymer surfaces at specified time points (@=significantly different from TCPS; *=significantly different from 1% agarose; and **=significantly different from 100% PCL and 1% agarose, or only to 100% PCL if located above the 1% agarose bar).

FIGS. 8A to 8E include confocal microscopy images of human coronary artery endothelial cells (hCAECs) 3 days post-seeding on (FIG. 8A) TCPS, (FIG. 8B) 100% PCL, (FIG. 8C) 96% PCL-04% ACPCL, (FIG. 8D) 89% PCL-11% ACPCL, and (FIG. 8E) 88% PCL-12% ACPCL.

FIG. 9A to 9C include images of an 88% PCL-12% ACPCL shape memory arterial bypass graft (FIG. 9A) in its original tubular shape, (FIG. 9B) after being heated, deformed, and fixed into its temporary, thread-like shape, and (FIG. 9C) after recovery of the original tubular shape at 37° C.

FIGS. 10A to 10E include schematics for a minimally-invasive bypass grafting of (FIG. 10A) an occluded blood vessel (e.g., double carotid artery ligation), showing (FIG. 10B) implantation and suturing of the SMP in its thread-like geometry, (FIG. 10C) functionalization by embedding in collagen hydrogel with C16 and AC-SDKP peptides, (FIG. 10D) recovery of the SMP's tubular original shape, and (FIG. 10E) blood perfusing through the tube and functional biomolecules that induces angiogenesis for regeneration and reperfusion of the occluded region over time.

FIGS. 11A-11C show that after 2 weeks, the very strong fluorescent signal in the “Polymer+Peptide” group from detection of fluorescent beads using fluorescence microangiography (FIG. 11A) indicates that blood was flowing through the tubular construct. There is little to no visible fluorescence in the other test groups (FIGS. 11B and 11C), signifying near-complete occlusion without this combination treatment.

FIGS. 12A to 12B include images of hematoxylin & eosin (H&E) staining after two weeks of in vivo grafting showing capillary connection between the polymer tube and native artery.

FIG. 13 includes a fluorescence microscopy image showing CD31 staining as a vascular endothelial cell and leukocyte marker in the “Polymer+Peptide” group after 2 weeks. Scale bar=200 μm.

FIG. 14 shows an example of n vascular external graft or support of the present invention, as well as optional features thereof, including shape memory properties and anti-neointimal therapeutic features.

FIGS. 15A to 15B demonstrate properties of x% PCL-y% ACPCL polymers. 15A. Three consecutive thermomechanical (TM) cycles with high, repeatable shape fixity and shape recovery and 15B macroscopic shape memory demonstrations illustrate excellent shape memory capabilities.

FIG. 16 shows an embodiment of the present invention and demonstrates mean stress distribution at the end-to-side Dacron graft-artery anastomosis. Stresses along sutures are approximately 8×larger than along the distal host artery. Similar results were obtained for the artery and vein grafts in this geometry.

FIG. 17 demonstrates that MK2i inhibits MAP-KAP Kinase II (MK2). MK2 is in the stress-activated protein kinase cascade. Stress, injury, TGFB, cytokines and lyso phosphatidic acid (LPA) activate p38 map kinase which in turn activates MK2. MK2 activates fibrotic pathways via LIM kinase and the small heat shock protein HSPB1 which leads to myofibroblast formation and deposition of ECM.

MK2 also activates hnRNPAO and TTP, transcription factors which lead to cytokine production. Thus, MK2i inhibits both fibrosis and inflammation, processes integral to neointimal formation.

FIG. 18 is a graph that shows MK2i effects intimal thickening. HSV rings cultured in RPMI medium (30% FBS) for 14 days either untreated (Control) or MK2i treated 2 hours prior to culture. Intimal thickening measured morphometrically. *p<0.01 (N=4-5).

FIG. 19 is a graph that shows the effect of MK2i on wall thickness in vivo. Mouse inferior vena cava to aorta interposition grafts were performed. Prior to implantation, grafts were incubated for 20 minutes in MK2i (100 μM). Weekly duplex ultrasound measurements suggest MK2i's effects were predominantly in the first week of treatment.

FIGS. 20A-20F show a 3D Printing Method to make prototypes. FIG. 20A) Positive mold design and FIG. 20B) print. FIG. 20C) (side view) Negative PDMS/glass and FIG. 20D) (top view). FIG. 20E) Porous 89% PCL-11% ACPCL. FIG. 20F) Final y-shape CAD design.

FIGS. 21A-21B show MK2i release from a depot gel layer on scaffolds with varying gel integrity. FIG. 21A. Schematic diagram of the poly (DOPA) coating and heparin immobilization on the adventitial face of SMP scaffold for unidirectional, sustained release of MK2i. FIG. 21B. The depot layer with the higher integrity (crosslinking density) releases MK2i (100 μM loading) at a more sustained rate than the depot layers with the lower integrities.

FIG. 22 shows a scheme for vascular access creation.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter includes compounds and methods for treating vascular conditions. In some embodiments the presently disclosed compounds include novel allyl-functionalized shape memory polymers (SMPs) that can be crosslinked via pendant allyl groups. In some embodiments the presently disclosed materials, such as vascular grafts, are comprised of the SMPs, and in certain embodiments include thermo-responsive SMPs that actuate at or near physiological temperature (e.g., about 37° C.). The present materials and grafts are advantageous because they can be relatively high in elastic recovery, easy to manufacture and program, low cost, compatible with vasculature, tunable, and/or biodegradable. Thus, embodiments of the present materials that possess some or all of these features are advantageous for manufacturing simple and minimally invasive implantable devices for various biomedical applications.

In this regard, the presently disclosed subject matter includes compounds that can form SMP materials. In some embodiments the compounds comprise a first monomer that is allyl-functionalized and crosslinkable and a second monomer that is not crosslinkable. In specific embodiments the first monomer is photocrosslinkable. The methods for making the present compounds are not particularly limited, and in some embodiments the compounds are made via a process that includes ring-opening polymerization.

Hemodialysis is the primary lifeline for patients with end-stage renal disease (ESRD), but arteriovenous graft (AVG) failure imposes significant morbidity, mortality, and financial impositions. Stenosis at the venous anastomosis ultimately leads to compromised blood flow, necessitating vascular interventions. Failure rates of 50% after 1 year and 75% after 2 years are reported in hemodialysis patients that utilize polytetrafluoroethylene (PTFE) dialysis grafts.

AVG failure remains an unmet clinical need. External mesh supports applied in other settings, such as to saphenous vein grafts in heart or peripheral bypass grafting surgeries, have been shown to inhibit neointimal formation. These materials had limited success in the hemodialysis setting because of geometric complexities at the venous anastomosis and complications such as infection and suture dehiscence. Embodiments of the present invention include mechanically compliant, moldable external supports that can be custom fit around each dialysis graft anastomosis without suturing to prevent neointimal formation. Devices of the present invention provide localized, sustained delivery of therapeutics with anti-neointimal effects to further abrogate neointimal formation.

Amelioration of AVG failure would significantly impact clinical outcomes and economic repercussions of hemodialysis patients. This proposal offers a unique plat form to advance adventitial drug delivery approaches and, if successful, could lead to therapeutic solutions in other clinical settings, such as coronary artery and peripheral bypass grafting surgeries.

Arteriovenous graft (AVG) failure imposes substantial morbidity, mortality, and financial impositions for end-stage renal disease (ESRD) patients undergoing hemodialysis. Stenosis at the venous anastomosis leads to compromised blood flow, necessitating repeated vascular interventions. AVG failure occurs ˜90% of the time at the venous anastomosis. Failure rates of 50% after 1 year and 75% after 2 years are reported in hemodialysis patients utilizing polytetrafluoroethylene (PTFE) AVGS.

There is no treatment available to effectively prevent AVG failure. Several approaches localizing treatment to the venous anastomosis have initially demonstrated promise, only to fail clinically due to adverse complications (e.g., infection, suture dehiscence) or lack of patency benefit. The closest-to-market approach, a sirolimus-eluting collagen membrane (Coll-™), is prone to infection because of sirolimus' immunosuppressant activities and long surgery times required for suturing.

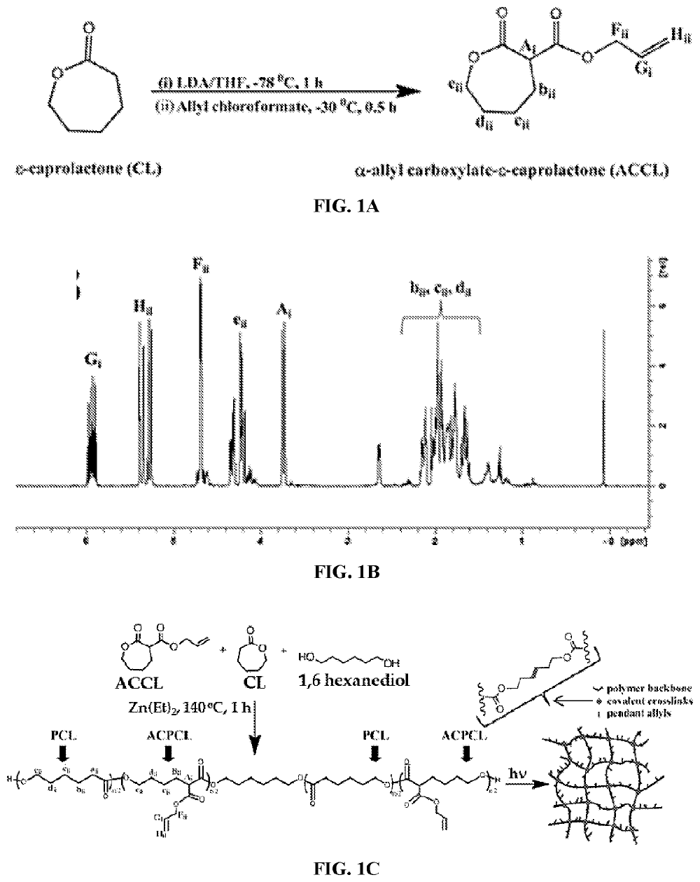

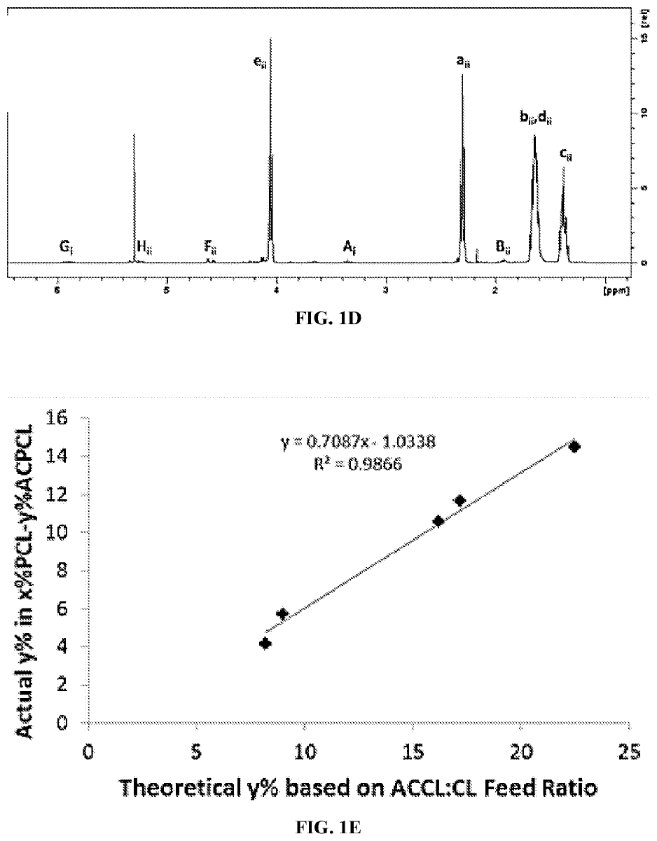

An embodiment of the present invention is a custom-fittable external support that does not require sutures and in further embodiments may elute a therapeutic such as an anti-neointimal, pleotropic peptide. The support can be custom fit around the venous anastomosis to prevent neointimal formation and associated AVG failure via promotion of outward instead of inward remodeling and localized, sustained delivery of the therapeutic (FIG. 1).

Kidney disease is the 9th leading cause of death in the US. In 2011. It was estimated that 31 million people have chronic kidney disease and 615,899 have kidney failure (i.e., end-stage renal disease: ESRD). ESRD patients require either transplants or dialysis to survive. The number of patients on hemodialysis was approximately 408,711 in 2012 and has grown by approximately 12, 632 every year since 2000.

PTFE AVGs are a common form of hemodialysis vascular access but fail at a rate of approximately 50% at 1 year and 75% after 2 years due primarily to neointimal formation. Once AVGs fail, interventional techniques (i.e., balloon angioplasty +/−stents) or re-do access surgeries are required. Patients with graft failure are approximately $87,895 more expensive to treat per patient-year, amounting to more than $4.8 billion in direct costs and growing every year.

The leading cause of failure is neointimal formation at the venous anastomosis triggered by venous responses to surgical injury from PTFE implantation, arterial flow, and other factors. These events lead to inflammation with phenotypic modulation, migration and proliferation of vascular smooth muscle cells (VSMCs); and subsequent deposition of excessive matrix proteins to form a neointima.

Systemic pharmacological approaches have exhibited little efficacy in preventing vein failure, indicating the need for more localized approaches. Past attempts involve treatment from the outer adventitial layer including: i) a gel foam loaded with allogenic endothelial cells (Vascugel™, Shire Pharmaceuticals) that was subsequently terminated due to a lack of patency benefit; ii) a collagen collar loaded with an adenoviral vector containing a vascular endothelial growth factor D gene (Trinam™, Ark Therapeutics Group) whose Phase III clinical trial was terminated due to “strategic reasons”, and iii) a paclitaxel-eluting ethylene vinyl acetate wrap (Vascular Wrap™, Angiotech Pharmaceuticals) whose Phase III clinical trial was terminated due to a higher infection rate in the paclitaxel-treated group.

As a closest-to-market approach, a sirolimus-eluting collagen membrane (Coll-™ Vascular Therapies) demonstrated safety and technical feasibility in a Phase I/II clinical trial. However, this trial was done with only 12 patients unrepresentative of the hemodialysis population (all Caucasian, only one diabetic, no common comorbidities such as coronary or peripheral arterial disease) and lacked a control group. Moreover, an elaborate suturing procedure was required to wrap the venous anastomosis, which not only increases surgery time and cost, but also increases the risk of suture dehiscence, patient discomfort and infection, especially for a more representative population. Sirolimus, an immunosuppressant, may also increase the risk of these adverse complications.

To address this long-felt need, embodiments of the present invention include a sutureless, custom-fittable external support that optionally elutes a pleotropic, non-immunosuppressive, anti-neointimal peptide. Combining novel shape memory polymers (SMPs) with the promising peptide should ultimately reduce neointimal formation, re-do operations and other adverse events for patients relying on hemodialysis, coronary artery bypass grafting (CABG), peripheral bypass grafting (PVBG), or other arteriovenous shunts to survive.

Thus, embodiments of the present invention include a new class of poly (ε-caprolactone) (PCL)-based SMPs, PCL-co-(α-allyl carboxylate -caprolactone) (x% PCL-y% ACPCL) [x% and y%:molar percentages], to fully address the design criteria established for external stenting of hemodialysis grafts.

Previously investigated polymers and shape memory alloys demonstrated promising ant-intimal hyperplasia effects in vein grafts in various CABG and PVBG preclinical models, but are difficult to apply to the variable, geometrically complex anastomoses encountered clinically, and require sutures.

Thermo-responsive SMPs address this issue. SMPs recover their original, permanent shape from a different, temporary shape by heating above a shape transition temperature (T) (e.g., melting T:Tₘ). Heating SMPs above their T, during hemodialysis access surgery enables facile molding of external supports around geometrically complex anastomoses without sutures or large incisions, thereby reducing surgery times and associated infection risks while completely obviating the risk of suture dehiscence.

One aspect of the invention is an implantable vascular graft. Embodiments include grafts that have at least one crosslinked polymer, with the polymers including a first monomer that is crosslinkable and a second monomer that is not crosslinkable. The grafts are capable of transforming between an original shape and an implanted shape.

Another aspect of the present invention is an implantable tissue supporting device, in the form of a biodegradable polymeric scaffold that surrounds a tissue, the polymeric scaffold comprising at least one crosslinked polymer, the polymer including: at least one monomer that is crosslinkable and/or at least one shape memory polymer; wherein the device is capable of transforming between an original shape and an implanted shape; and wherein the device is mechanically compliant at from about 20 to about 50° C.

In embodiments of the aforementioned aspects of the invention, the first monomer is allyl functionalized and includes an allyl carboxylate group. Additionally, the first monomer, the second monomer, or both are an ester. In other embodiments, the first monomer, the second monomer, or both include ε-caprolactone (CL). Additionally, the plurality of crosslinked polymers may include a poly (ε-caprolactone)-co-α-allyl carboxylate E-caprolactone) polymer. In other embodiments, the plurality of crosslinked polymers may include about 1 mol % to about 30 mol % of the first monomer. In other embodiments, the plurality of crosslinked polymers includes a shape transition temperature from about 20° C. to about 50° C.

Embodiments of the present invention can be configured to transform from the original shape to the transplanted shape when heated above a shape transition temperature of the plurality of crosslinked polymers. The original shape may be a compressed form of the transplanted shape. The original shape may be a thread, a sheet, tubular shape, a shape corresponding to a blood vessel, a vascular patch, a vascular bypass graft, a vascular stent, and combinations thereof. The transplanted shape may be a shape corresponding to a blood vessel, a vascular patch, a vascular bypass graft, a vascular stent, and combinations thereof.

Embodiments of the present invention may option ally further include a bioactive agent. The bioactive agent may be at least one of a pleotropic agent, growth factor, peptide, nucleic acid, pharmacological agent, MK2 inhibitor, anti-proliferative agent, anti-migratory agent, anti-inflammatory agent, or anti-fibrotic agent. The bioactive agent may also be at least one of rapamycin, tacrolimus, paclitaxel, marimastat, dexamethasone, pioglitazone, AZX, or cilistazol.

Embodiments of the preset invention may have 50-100% shape fixity, and/or 50-100% shape recovery. The Young's modulus at 37° C. may be about 0.05-200 MPa.

As indicated above, embodiments of the present invention surrounds a tissue. In preferred embodiments, the tissue may be a vein or artery. Also, the embodiments maybe external to the vein or artery. Preferably embodiments may be external to a vascular graft anastomosis.

Once implanted, embodiments of the present invention may form a seamless and sutureless sheath. The sheath is mesh or netting. Additionally, once implanted, preferably embodiments of the invention have resilient radial expression in a manner that mimics the compliance properties of said tissue. They may be deformable by at least one of stretching or bending along its length to conform to the shape of the tissue.

Embodiments of the present invention afford the unique capability to provide a custom fit for each anastomosis. This spatial control between the stent and vein critically affects adventitial micro vessel formation and outward remodeling that can mitigate neointimal formation. It can also help to minimize asymmetric wall thickening that causes turbulent, irregular flow and subsequent thrombosis and hyperplasia, especially around anastomoses (see FIG. 14).

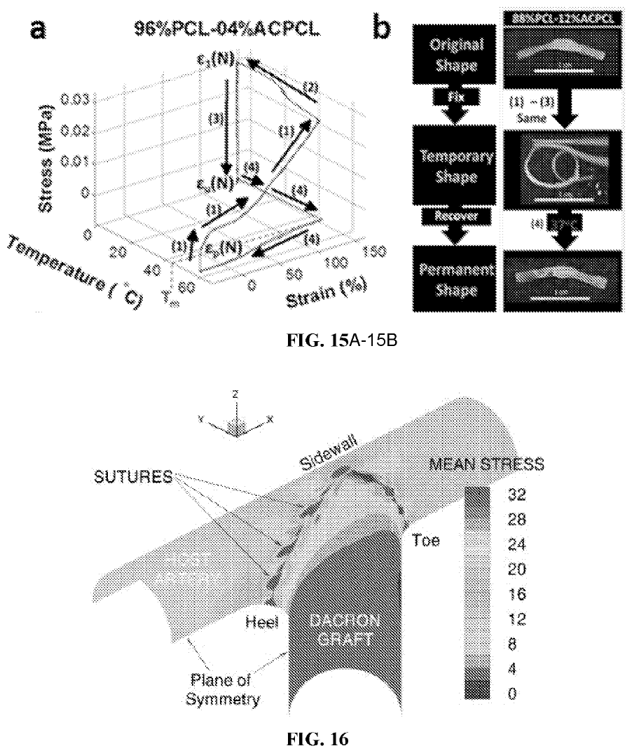

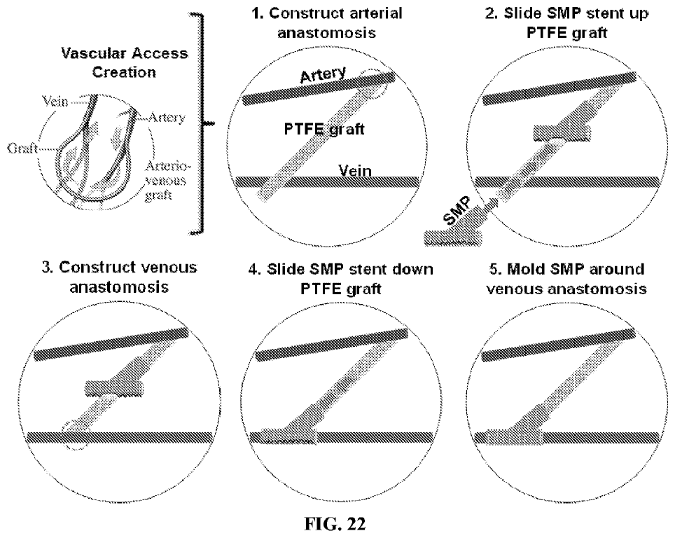

Custom fitting at vascular access operating temperatures (e.g., 28-37° C.) is made possible because copolymerizing ε-caprolactone (CL) with novel CL derivative α-allyl carboxylate-ε-caprolactone (ACCL) produces a polymer library with T_{y}'s from 28-43° C. and exceptional shape memory properties (FIG. 15). Given their shape memory capabilities at 37° C. the geometry of external supports can be custom tailored by the surgeon with relative ease to fit the asymmetric distal anastomosis (See FIG. 22).

This unique copolymerization format also enables fine-tuning of thermomechanical properties such that SMP stents can be fabricated with artery-mimetic mechanical properties. This is important because compliance mismatch between the vein and synthetic graft or artery is another factor involved in neointimal formation. For example, a 68% decrease in mechanical compliance from a blood vessel to a graft, equivalent to transitioning from an artery to Dacron, results in a 40% increase in mean anastomotic stress along suture lines and subsequent neointimal formation in an end-to-side geometry (FIG. 16). The material properties of this SMP library are highly tunable as the molar composition, molecular weight, and crosslinking density all can be varied to render a wide range of elastic moduli (1-100 MPa at 37° C.), which is still stiffer than arteries (˜1.3 MPa). This SMP library therefore provides the unique opportunity to generate mechanically compliant, custom fittable external supports.

Porosity is also critical in fostering adventitial micro vessel formation and can be controlled in stent fabrication.

Embodiments of the present invention may be slowly biodegradable (>1 year) and bioresorbable, ensuring that their mechanical properties are maintained until vein remodeling is stabilized while being ultimately resorbed to avoid potential long-term complications.

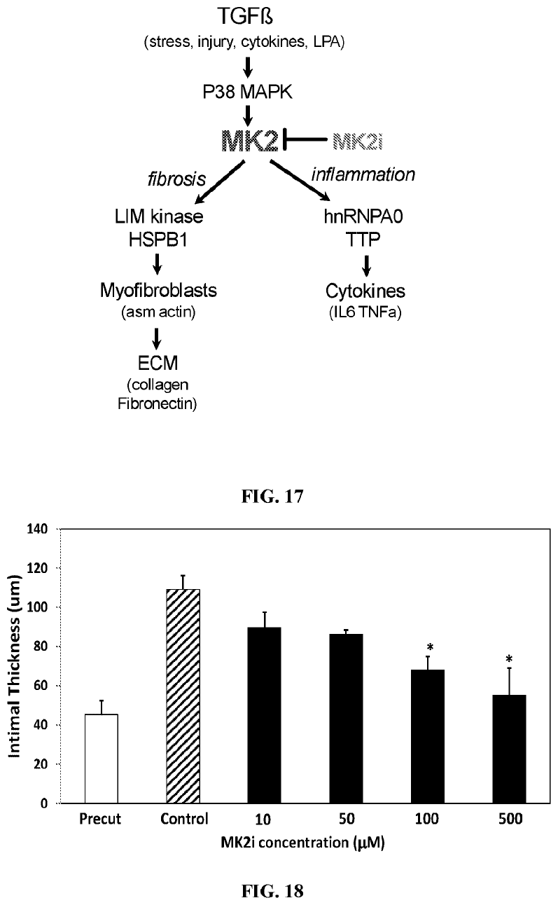

As stated above, embodiments of the present invention include the addition of an anti-neointimal peptide. With its anti-fibrotic and anti-inflammatory properties (FIG. 17), a peptide inhibitor of MK2 (MK2i) has shown promise as an agent to prevent neointimal formation. MK2 is down stream of the TGFB-p38 stress-activated protein kinase pathway, conferring specificity and limiting off target toxicity.

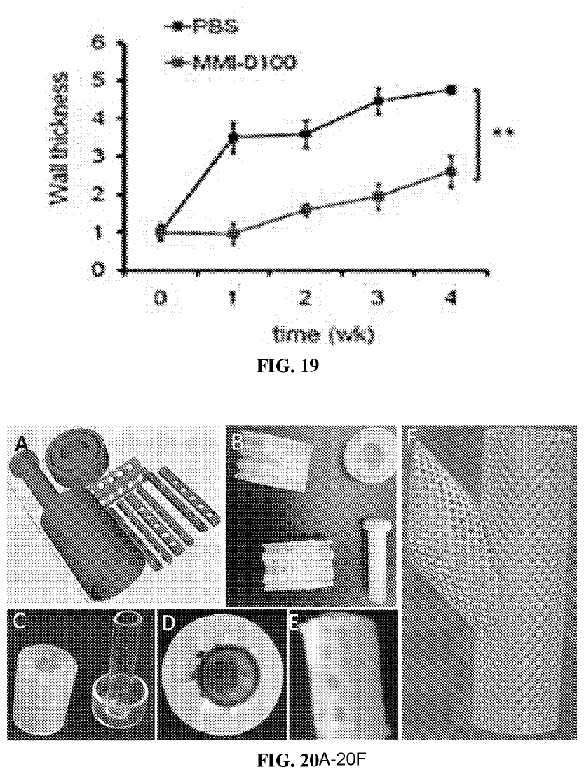

MK2i has been shown to inhibit VSMC proliferation, migration, and most importantly, synthetic phenotypic modulation. Treatment of human saphenous vein (HSV) with MK2i in an ex vivo organ culture model led to decreases in intimal thickening (FIG. 18). In an in vivo murine inferior vena cava interposition into the aorta model, a single, 20-minute ex vivo MK2i treatment of the vein graft prior to implantation decreased wall thickness by 72% at 28 days (FIG. 19). These data indicate MK2i as one of the most comprehensive therapeutic approaches to prevent the constellation of events leading to neointimal formation. More over, an external support could further abrogate neointial formation by prolonging its effects (FIG. 19).

The ratio of the first monomer to the second monomer is also not particularly limited. In some embodiments the compound is comprised of about 1 mol %, 5 mol %, 10 mol %, 15 mol %, 20 mol %, 25 mol %, 30 mol %, 35 mol %, 40 mol %, 45 mol %, or 50 mol % of the first monomer. In other embodiments the compound is comprised of about 1 mol % to about 50 mol % of the first monomer, about 1 mol % to about 30 mol % of the first monomer, or about 1 mol % to about 15 mol % of the first monomer. In such embodiments the remainder of the polymer can be comprised of the second monomer.

In some embodiments the first monomer, the second monomer, or both include an ester. The term “ester” as used herein is represented by a formula R₁OC(O)R₂ or R₁C(O)OR₂, wherein R₁, and R₂, can be independently selected from, but are not limited to, an optionally substituted alkyl, alkenyl, alkynyl, or the like. The term ester is inclusive of “polyester,” or compounds comprising two or more ester groups.

In some embodiments the first monomer that is allyl-functionalized includes an allyl carboxylate group. In such embodiments, the monomer may include a carboxylate group that is then functionalized with an allyl group, or the monomer may be functionalized with the carboxylate allyl group. The carboxylate allyl group described herein can be represented by the following formula:

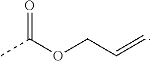

In some embodiments the first monomer, the second monomer, or both ε-caprolactone (CL) and/or derivatives thereof. For instance, the first monomer including E-caprolactone can include an α-allyl carboxylate ε-caprolactone (ACCL) monomer. In some embodiments the compounds are based on polycaprolactone (PCL) because PCL has desirable properties for vascular applications, including biocompatibility, suitable rates of biodegradability, and mechanical compliance. Thus, in certain embodiments the compound includes a poly(ε-caprolactone)-co-(α-allyl carboxylate ε-caprolactone) copolymer (PCL-ACPCL), and some embodiments of the present compounds can include the following formula:

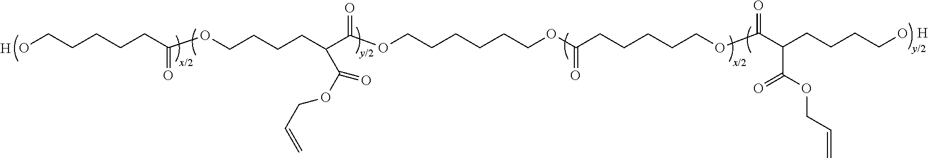

wherein x and y are integers having no particular limitation. Embodiments of the present polymers can also be characterized as x% poly(ε-caprolactone)-co-y% α-allyl carboxy late ε-caprolactone) (x% PCL-y% ACPCL) wherein x% and y% correspond to molar ratios and have no particular limitation.

In some embodiments of the compound is a block copolymer. A “block” copolymer refers to a structure comprising one or more sub-combination of constitutional or monomeric units. In some embodiments, constitutional units are derived via additional processes from one or more polymerizable monomers. There is no limitation on the number of blocks, and in each block the constitutional units may be disposed in a purely random, an alternating random, a regular alternating, a regular block, or a random block configuration unless expressly stated to be otherwise.

As mentioned above, the present compounds can include allyl-functionalized monomers that are crosslinkable. The terms “crosslinkable,” “crosslink,” and the like are used here to refer to an attachment of one portion of a polymer chain to a portion of the same polymer chain or a portion of another polymer chain by chemical bonds that join certain atom(s) of the polymer chain(s). Exemplary chemical bonds that can form crosslinks include covalent bonds and hydrogen bonds as well as hydrophobic, hydrophilic, ionic or electrostatic interactions. In some instances covalently-crosslinked SMP materials exhibit superior shape memory properties and thermal stability when com pared to SMP materials crosslinked by non-covalent bonds.

Cross-linking can be effected naturally and artificially. For instance, in some embodiments the first monomer is photocrosslinkable, where the term “photocrosslink” and the like is used herein to refer to crosslinks that are formed upon being exposed to electromagnetic radiation, such as visible light and/or ultraviolet radiation. In some embodiments photocrosslinks can be formed by exposure to ultra violet light having a wavelength of about 100 nm to about 300 nm. The terms “crosslink” and the like as used herein can be inclusive of the terms “photocrosslink” and the like.

In some embodiments the allyl-functionalized monomer includes a pendant allyl-including group (e.g., carboxylate allyl group) that can crosslink. In some embodiments the allyl-including group can photocrosslink to another allyl-including group of the same compound or another compound.

In some embodiments the present compounds can further comprise a bioactive agent. The term “bioactive agent” is used herein to refer to compounds or entities that alter, promote, speed, prolong, inhibit, activate, or otherwise affect biological or chemical events in a subject (e.g., a human). The manner in which the bioactive agent is incorporated into a compounds is not particularly limited. In some embodiment the bioactive agent can be incorporated (e.g., mixed with) the compound. In some embodiments the bioactive agent can be covalently bound to an allyl-including group of the first monomer via thiol-ene click chemistry.

Exemplary bioactive agents may include, but are not limited to, anti-cancer substances, antibiotics, immunosuppressants, anti-viral agents, enzyme inhibitors, neurotoxins, opioids, hypnotics, anti-histamines, lubricants, tranquilizers, anti-convulsant, muscle relaxants, anti-spasmodics and muscle contractants including channel blockers, growth factors, miotics and anti-cholinergic, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, cell response modifiers, cells, peptides, which as used herein includes polypeptides, viruses, and vaccines.

In some embodiments the present compounds are biocompatible. Indeed, certain embodiments the present compounds and grafts are more biocompatible with endothelial cells (ECs) than 100% PCL, as indicated by higher levels of long-term cell viability and healthy cell morphologies. The term “biocompatible” as used herein is intended to describe a characteristic of substances that do not typically induce undesirable or adverse side effects when administered in vivo. For example, biocompatible substances may not induce side effects such as significant inflammation and/or acute rejection. It will be recognized that “biocompatibility” is a relative term, and some side effects can be expected even for some substances that are biocompatible. In some embodiments, a biocompatible substance does not induce irreversible side effects, and in some embodiments a substance is biocompatible if it does not induce long term side effects. One test to determine substance is to measure whether cells die upon being exposed a material in vitro. For instance, a biocompatible compound or graft may cause less than about 30%, 20%, 10%, or 5% cell death.

Additionally or alternatively, some embodiments of the present compounds are biodegradable. The term “biodegradable” as used herein describes a characteristic of substances that degrade under physiological conditions to form a product that can be metabolized or excreted without damage to the subject. In certain embodiments, the product is metabolized or excreted without permanent damage to the subject. Biodegradable substances also include substances that are broken down within cells. Degradation may occur by hydrolysis, oxidation, enzymatic processes, phagocytosis, other processes, and combinations thereof. Degradation rates for substances can vary, and may be on the order of hours, days, weeks, months, or years, depending on the material.

Embodiments of the presently-disclosed compounds can further comprise additional functional groups and/or monomers to impart desired characteristics upon the compounds. The addition of functional groups or monomers to the compounds can impart desired functionalities to the compounds and/or affect the melting temperature of the compounds. Thus, certain functional groups or monomers can be incorporated into a compound in order to tune the thermo-mechanical characteristics of the compounds.

The presently-disclosed subject matter also includes shape memory polymer (SMP) materials comprised of any of the presently-disclosed compounds. In some instances the materials are utilized to form grafts, such as vascular grafts for a blood vessel (e.g., vein, artery). Exemplary vascular grafts can include a plurality of crosslinked polymers, the polymers including a first monomer that is allyl-functionalized and crosslinkable and a second monomer that not crosslinkable, and the graft can be capable of transforming between a temporary shape and an original shape.

The term “implanted shape” refers to a shape that has been given to a material by exerting a force on the material and/or exposing the material to certain temperatures (i.e., programming step). While the material can retain its temporary shape for any length of time, the shape is referred to as being temporary because the shape exists only when external forces exerted on the material. Furthermore, in some embodiments the materials can lose their temporary shape when exposed to a temperature above a melting temperature of the material, as described below.

The term “original shape” refers to a shape of the material when the polymers of the material are in their native, pre-implanted, unstrained state. Once a material is in its original shape, a material will generally retain the original shape unless an external forces or the like is applied to the material. Some embodiments of materials revert to and/or retain an original shape when exposed in a physically unstressed state to a temperature above a melting temperature of the material (i.e., recovery step). Crosslinks between the plurality of polymers that comprise the materials, either chemical or physical in nature, help prevent irreversible, plastic deformation during programming and recovery steps.

There are no particular limitations on what shapes can be assumed by the material in its temporary shape or its original shape. In some embodiments temporary shape is selected from a thread, a sheet, tubular shape, a shape corresponding to a blood vessel, a vascular patch, a vascular bypass graft, a vascular stent, and combinations thereof. Likewise, in some embodiments the original shape can be selected from a thread, a sheet, tubular shape, a shape corresponding to a blood vessel, a vascular patch, a vascular bypass graft, a vascular stent, and combinations thereof. As discussed further below, certain shapes can be advantageous for certain therapeutic uses of the present materials.

Embodiments of the present materials can thus be categorized as thermomechanical SMPs, whereby the polymers can exhibit a transition from a temporary shape to an original shape when transitioning above and/or below a melting temperature of the compounds. For instance, a material may initially have an original shape, and a temporary shape can be induced by heating the material above its melting temperature while exerting a force on the material that molds or bends the material into a desired temporary shape. The material can retain its temporary shape if it is then cooled to a temperature below the melting point of the material while holding the material in the temporary shape, and the material can substantially retain this temporary shape so long as it is kept at a temperature below the melting temperature of the material. Subsequently, the material can revert to its original shape by heating the material to a temperature above its melting temperature.

The present compounds and materials comprising the present compounds can include wide range of melting temperatures. In some embodiments the compounds and materials comprising the compounds include a melting temperature of about 20° C. to about 50° C. including melting temperatures of about 20° C., 25° C., 30° C. 35° C. 40° C. 45° C. and 50° C. In some embodiments the compounds and materials comprise a melting temperature that is at or substantially near physiological temperature (e.g., about 37° C.) so that the materials may experience a switch-like shape transition when implanted into a subject. The present materials can also include relatively high elastic recovery. In some embodiments the present materials include a strain recovery rate (Rr) and/or strain fixity rate (Rf) of 90% or more, and in some embodiments Rr and Rf can independently be about 91%, 92%, 93%, 94%, 95%, 96%, 97%, 98%, or 99% or more. The present materials can also possess qualities that make them similar to and therefore appropriate for use in conjunction with and/or as a replacement for blood vessels. For instance, some embodiments of materials have compliant and ductile qualities that are suitable for use with vasculature. Some embodiment scan also include elastic moduli of about 1.0 to about 200.0 MPa at 37° C. which can be suitable for certain vascular applications.

The shape memory properties of the present materials can be tuned by modifying the present compounds. The melting temperature and other properties of the materials can be altered by modifying the compounds in a manner that affects the allyl groups of the allyl-functionalized first monomer. Without being bound by theory or mechanism, this is due to the fact that the allyl of a compound can affect the crystallinity and spacing of netpoints of the compound and any materials comprising the compounds. The molar concentration of the first monomer and/or the concentration and arrangement of allyl groups on the first monomer can therefore offer efficient means for tuning the thermomechanical, shape memory, and biological functions of the present materials. In some instances the properties of certain embodied materials can be further tuned through alteration of the molecular weight or gel content of the materials.

The present compounds and materials described herein therefore have the superior and unexpected advantage of having tunable properties, and in some instances can be tuned to have physiologically relevant melting temperatures. Methods for tuning the properties of the compounds and materials include, but are not limited to, varying the molar concentration of the allyl-functionalized first monomer in the polymer, varying the concentration of allyl groups in the allyl-functionalized first monomer, and varying the size and molecular weight of the first monomer, the second monomer, or other monomers in the polymers, or combinations thereof. In certain embodiments can be tuned to mimic a range of soft tissues.

The presently-disclosed subject matter further includes method for treating a vascular conditions. In some embodiments the method comprises administering a vascular graft in a temporary shape to a subject in need thereof, the graft comprising a plurality crosslinked polymers that include a first monomer that is allyl-functionalized and crosslinkable and a second monomer that not crosslinkable. The embodied methods further comprise a step of allowing the vascular graft to transform from the temporary shape to an original shape. The transformation from a temporary shape to an original shape can be initiated by heating the graft above the melting point of the plurality of polymers, and in some embodiments the heating is done passively from heat that is emitted from the subject.

The step of administering the graft can include coupling the graft to a blood vessel of interest. As used herein, the term “couple” and the like refers to the attachment of the graft to a blood vessel by any means. In some instances coupling refers to wrapping a sheet-like graft around a blood vessel. In other instances coupling refers to suturing a thread-like graft to a blood vessel. In yet other instances coupling can refer to inserting a blood vessel through an opening of a tubular graft. Thus, the term “couple” broadly refers to a multitude of methods of configuring a graft in relation to a blood vessel or other treatment target.

The terms “treatment” or “treating” refer to the medical management of a subject with the intent to cure, ameliorate, stabilize, or prevent a disease, pathological condition. The term “condition” is inclusive of diseases, disorders, and the like. “Treatment” includes active treatment, that is, treatment directed specifically toward the improvement of a condition, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Furthermore, the terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

Vascular conditions that can be treated by the present grafts include, but are not limited to, strokes, aneurisms, ischemic vessels, hemorrhages, occlusions, ruptured vessels, rupture-prone vessels, stenosis, atherosclerosis, peripheral artery disease, an arteriovenous fistula, or a combination thereof. Those of ordinary skill in the art upon reviewing this application will appreciate other vascular conditions as well as non-vascular conditions that can be treated with the present materials.

The graft can be implanted in its temporary shape or its original shape. In the event that the graft is implanted in a temporary shape, embodiments of the treatment methods can further include, before the administering step, a step of cooling the graft in a temporary shape to a temperature below the melting temperature.

The mechanical and thermal properties of the present grafts can be tuned within this system to more closely match that of the native blood vessels. In some embodiments the present grafts can include an elasticity that is akin to that of a native artery. This biomimicry can allow the present grafts to achieve superior results when compared to vein grafts or other synthetic grafts. For example, veins are not designed for and do not perform well under sinusoidal flow conditions typically experienced by arteries, and also do not comprise a muscle layer akin to that of arteries. Consequently, vein grafts, such as saphenous vein grafts, can experience atherosclerosis, intimal hyperplasia, thrombosis, and restenosis. Furthermore, the process of grafting and processing a vein can itself cause ischemic damage to the vein. On the other hand, by virtue being elastic and mimicking other mechanical properties of arteries, the present grafts can be utilized as arterial grafts with fewer or none of the negative side effects typically experienced by vein grafts.

Additionally, surgical procedures for treating vascular conditions, such as conventional bypass surgery, are typically highly-invasive, which can prolong patient recovery and hospitalization times and limit treatment options for those with arterial occlusions. However, the embodiments of the present grafts can include a temporary shape that facilitates the procedure and render it less invasive. For example, in some embodiments grafts can be programmed into a thin thread-like temporary shape that permits administration via small bore catheters and can permit for manipulation of the graft alongside an artery. Alternatively, exemplary grafts can be tunneled along an artery via attachment to a tunneling device. Those of ordinary skill will appreciate other temporary shapes and methods for administering the grafts that can reduce the invasive nature of procedures for treating vascular conditions.

In specific embodiments the grafts can be utilized for bypass procedures. In some embodiments the graft includes an original shape that is a stent, which often takes an elongated tubular form. The graft can be coupled to the outside of a vein graft by wrapping or placing the graft around vein graft. This configuration can improve the adaptation of the vein to the high pressure, high flow environment of the arterial circulation. In such embodiments the graft can include a temporary shape of a sheet, such that the graft can be administered by coupling (i.e., wrapping) the sheet around the vein graft and subsequently allowing the graft to transition to its original stent shape in order to support the vein graft.

Some embodiments of the present treatment methods also provide bypass procedures that do not require transection of a native artery. For instance, the graft can include a temporary shape that is a thread shape (i.e., elongated thread) for easy insertion of the graft into the subject as well as easy manipulation of the graft long the artery. The graft can then be coupled to the artery by ligating it to the artery with sutures or the like, and subsequently the graft can transform to its original vascular bypass graft shape. Subsequently, capillary ingrowth can be achieved from the artery into the adjacent graft such that the occluded region section of the adjacent artery can be regenerated and reperfused over time. Additionally, in some embodiments the graft can include and/or can be administered in conjunction with bioactive agents (e.g., peptides, growth factors, etc.) that can facilitate angiogenesis.

Treatment can also refer to the placing a graft within or on a blood vessel that has ruptured or that is prone to rupture. The graft can then include an original shape of a blood vessel patch that closes and protects the rupture or potential rupture.

The presently-disclosed compounds and grafts therefore present several advantages for methods of treating vascular conditions. First, the grafts can include an original shape that provides for a custom-fit graft that avoids flow mediated thrombosis and hyperplasia. The ability to customize the original shape of the graft also makes it suitable for unusual vasculature, such as branched arteries, as well as for treating other non-vascular conditions. The ability to customize the temporary shape also permits the present grafts to achieve robust and facile surgical placement via minimally invasive techniques.

Once implanted, the present grafts can offer mechanical compliance that withstands blood vessel pulsation similar to an artery. Further still, embodiments of the present grafts can be biocompatible and, optionally, can exhibit biodegradable characteristics that are sufficiently slow to permit healing of the vasculature. The present grafts can also have a porosity that promotes microvascular growth to repair damaged vessel tissue. The present grafts can therefore provide treatment methods that are easily implemented, cost effective, and less invasive to the subject.

Additionally, presently-disclosed subject matter further includes a kit that can include a material comprised of an embodiment of the present compounds, packaged together with a device useful for administration of the material. As will be recognized by those or ordinary skill in the art, the appropriate administration-aiding devices will depend on the temporary shape of a graft and/or the desired administration site.

### EXAMPLES

The presently-disclosed subject matter is further illustrated by the following specific but non-limiting examples. The following examples may include compilations of data that are representative of data gathered at various times during the course of development and experimentation related to the presently-disclosed subject matter.

### Example 1

This example describes the synthesis and characterization of an exemplary x% PCL-y% ACPCL copolymer library. To prepare this copolymer library, a novel α-allyl carboxylate ε-caprolactone (ACCL) monomer was first synthesized in a single reaction by lithium diisopropyl amine (LDA)-mediated carbanion formation at the a-carbon of E-caprolactone (CL) and subsequent addition of allyl chloroformate (FIG. 1A). More specifically, in a 250 mL round bottom flask, distilled CL (13.9 mL, 125 mmol) was added dropwise to LDA (125 mL of 2 M in THF/n-heptane/ethylbenzene, 250 mmol) in anhydrous THF (200 mL) at −78° C. After 1 hour, the temperature was raised to −30° C. and allyl chloroformate (13.3 mL, 125 mmol) was added dropwise. Thirty minutes later, the temperature was raised to 0° C. and quenched with saturated NH₄Cl (30 mL). The crude ACCL was diluted in H₂O (100 mL), extracted with ethyl acetate (300 mL×3), dried with Na₂SO₄ filtered, evaporated, and purified by column chromatography using Silica Gel Premium Rf (Sorbent Technologies, Norcross, Ga.) with 10% ethyl acetate in hexanes. Yield: 58% (14.3 g, 72 mmol). ¹H-NMR confirmed formation of the desired ACCL product, as indicated by characteristic allyl (5.92 (Gᵢ), 5.31 (Hᵢᵢ) and 4.63 (Fᵢᵢ) ppm) and CL peaks (FIG. 1B).

Ring-opening (co) polymerization (ROP) of ACCL with CL using a diethylzinc catalyst and 1,6-hexanediol initiator generated a library of novel x% PCL-y% ACPCL (x and y:molar ratio) copolymers with y=4.16-14.50% as determined by the ratio of allylic CH protons (G₁, 6=5.92 ppm) to CH₂ protons at the ε-carbon of PCL and ACPCL units (Eᵢᵢ, δ=4.15 ppm) (FIGS. 1C and 1D, Table 2). To form these polymers, varying molar ratios of dried ACCL and CL (100 mmol total) were introduced to a pre-dried test tube containing 1,6-hexanediol (0.5 mmol). The polymerization mixture was degassed with two freeze-purge-thaw cycles, submerged in a 140° C. oil bath, and catalyzed with drop wise addition of Zn (Et)₂ (1 mmol, 15 wt % in toluene) for 1 hour. The solution was precipitated in cold diethyl ether and dried under vacuum.

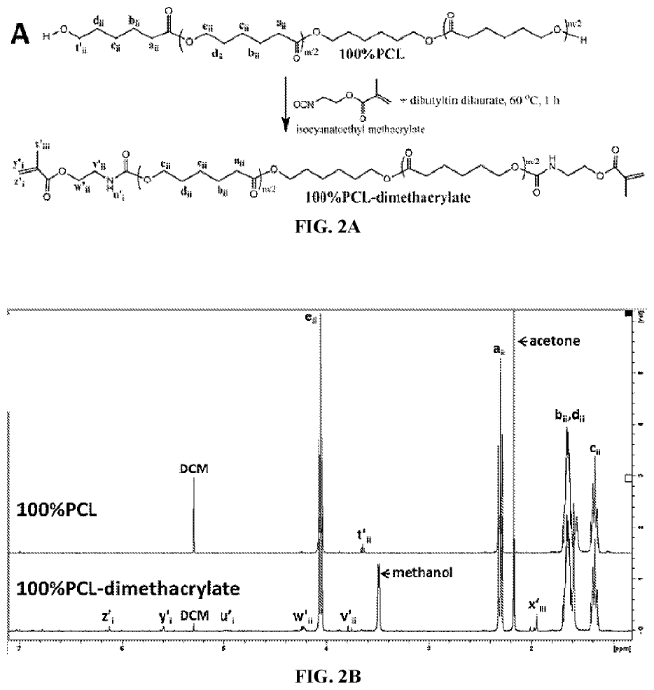

As a control, 100% PCL (Table 2, Mₙ=11300 Da, PDI=1.54) was similarly synthesized (Table 2, M,=11628 Da, PDI=1.41) by adding 2-isocyanatoethyl methacrylate (0.22 g, 1.42 mmol) to 100% PCL (1.0 g, 86.0 μmol) in anhydrous THF (20 mL) in a 100 mL round-bottom flask. The reaction mixture was heated to 60° C. and catalyzed with dibutyltin dilaurate (10 μL, 17 nmol) for 1 hour. The product was washed with 100% hexanes and 90% hexanel 10% methanol, then dried under vacuum. The terminal hydroxyl-to-methacrylate conversion rate, or degree of methacrylation (D_{M}), was calculated by summing the normalized methacrylate proton integrals from 6.12 (I_{6.12}) and 5.61 ppm (I_{5.61}) peaks for 100% PCL-dimethacrylate, and then dividing by the normalized integral from the CH₂ protons adjacent to the terminal hydroxyls for unmodified 100% PCL at 3.66 ppm (I_{2.66,notfunc}). The PCL exhibited a terminal hydroxyl-to-methacrylate conversion (D_{M}) of 90.5% (FIG. 2).

Allylic compounds attained were lower than the ACCL:CL feed ratios due to lower reactivity of the ACCL monomer (Table 2, FIG. 1E). Molecular weight (Mₙ=12-19 kDa, polydispersity index (PDI)=1.78-2.50) was controlled by the 1,6-hexanediol initiator:total monomer ratio but was also influenced by the feed ratio of the less reactive ACCL monomer. The higher PDIs and lower yields (22.6-56.6%) attained for these copolymers may be due to transesterification reactions involving both the polyester backbone and pendant allyl carboxylates. There is an inverse relationship between thermal properties and allyl composition, possibly because ACPCL disrupts PCL crystallinity, thereby lowering the Tₘ and percent crystallinity (X_{c}) (Table 2).

TABLE 1
Characterization of x % PCL-y % ACPCL copolymers

| | y % ACPCL | | | | | | | | |
|---|---|---|---|---|---|---|---|---|---|
| | Theoretical | Actual | | | | | | | |
| | y | y | Yield | | Mₙ | M_{w} | PDI | Tₘ | X |
| Copolymer | [%] | [%]^{a)} | [%] | Initiator:Monomer | [Da]^{b)} | [Da]^{b)} | [M_{w}/Mₙ] | [° C.] | [%]^{a)} |
| 100% PCL | 0 | 0 | 86.2 | 1:100 | 11300 | 17368 | 1.54 | 53.0 ± 0.2 | 56.6 ± 1.5 |
| 100% PCL- | 0 | 0 | N/A | N/A | 11628 | 16417 | 1.41 | 50.7 ± 0.5 | 45.8 ± 1.9 |
| dimethacrylate | | | | | | | | | |
| 96% PCL-04% ACPCL | 8.2 | 4.16 | 44.8 | 1:200 | 15060 | 26870 | 1.78 | 45.9 ± 0.3 | 41.6 ± 1.2 |
| 94% PCL-06% ACPCL | 9.0 | 5.74 | 38.3 | 1:200 | 16546 | 39050 | 2.36 | 47.1 ± 0.1 | 36.1 ± 0.5 |
| 89% PCL-11% ACPCL | 16.2 | 10.58 | 39.8 | 1:200 | 13627 | 34049 | 2.50 | 39.1 ± 0.3 | 30.4 ± 0.7 |
| 88% PCL-12% ACPCL | 17.2 | 11.66 | 22.6 | 1:315 | 19087 | 36430 | 1.91 | 41.6 ± 0.2 | 31.1 ± 0.7 |
| 85% PCL-15% ACPCL | 22.5 | 14.50 | 56.6 | 1:200 | 12095 | 28931 | 2.39 | 32.5 ± 0.4 | 24.4 ± 0.9 |

^{a)}y % ACPCL was determined by the ratio of the 5.90 ppm integral, I5.90, to the 4.15 ppm integral, I4.15:y % ACFCL = 2 × I5.90/I4.15 × 100%.;
^{b)}Molecular weight properties were determined by gel permeation chromatography against PMMA standards (Agilent Technologies, Inc., Santa Clara, CA) using a Phenogel 10E3A column (Phenomenex Inc., Torrance, CA) in THF.
^{c)}X_{G} = ΔHₘ/ΔHₘ^{o} × 100%, where ΔHₘ^{σ} = 139.5 J/g, the enthalpy of fusionfor 100% crystalline PCL.

### Example 2

This Example describes the preparation and characterization of crosslinked x% PCL-y% ACPCL and 100% PCL-dimethacrylate SMP films using the polymers synthesized in Example 1. A subset of x% PCL-y% ACPCL copolymers and the 100% PCL-dimethacrylate control were photocrosslinked to create the shape memory effect and evaluated in terms of gel content, thermal, mechanical, and shape memory properties. The crosslinked x% PCL-y% ACPCL and 100% PCL-dimethacrylate SMP films of uniform thickness (0.2-0.3 mm) were produced from a 10 wt % polymer solution containing 3 wt % 2,2-dimethoxy-2-phenylacetophenone via a thin film applicator (Precision Gage & Tool, Co., Dayton, Ohio) and 365 nm irradiation (4.89 J cm⁻², 18.1 mW cm⁻²) with a Novacure 2100 Spot Curing System (Exfo Photonic Solutions, Inc., Mississauga, Ontario, Canada). After drying, samples were incubated in DCM for 2 days to determine gel content. Thermal proper ties were measured on a TA Instruments (New Castle, Del.) Q1000 differential scanning calorimeter. Mechanical and shape memory properties were determined using a TA Instruments Q2000 dynamic mechanical analyzer in tensile mode.

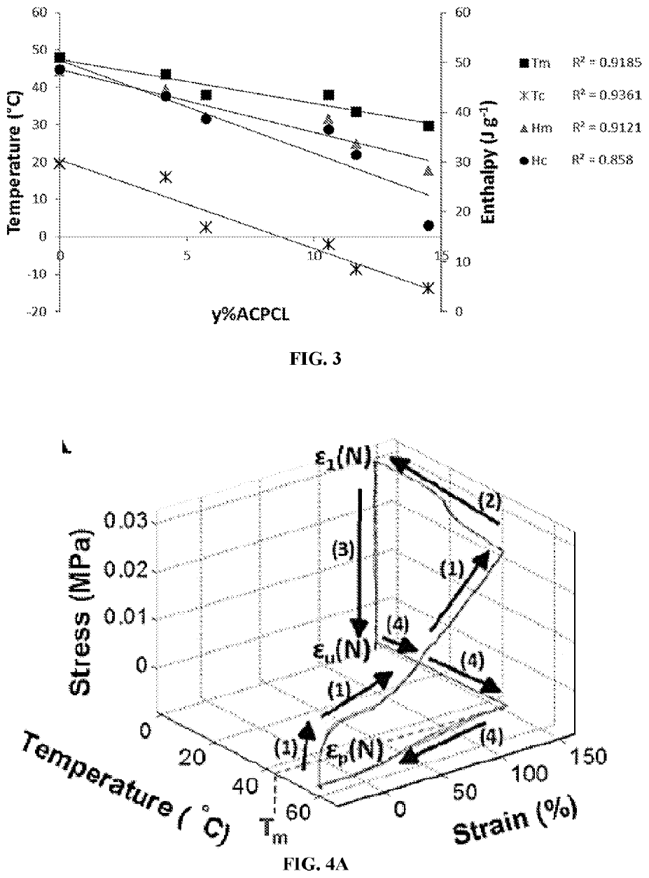

It was desired to produce SMPs with Tₘs both slightly above and below 37° C. as surgical preferences for the onset of shape recovery depend on the particular biomedical application. In order to be used for various vascular applications, it was also desired that the SMP library exhibits tunable mechanical properties, with sufficient compliance and extensibility. Moreover, complete and repeatable shape recovery with an on-off “switch-like” response to small temperature changes is sought after in order to tightly control shape memory behavior and preserve implant integrity and function following shape programming and recovery. Gel content (X_{G}) relates to the percent crosslinking of the material, and in some SMP networks a minimum X_{G} of 10% to 30% is required to achieve the shape memory effect. After photocrosslinking (365 nm, 4.89 J cm⁻², 18.1 mW cm⁻²), the X_{G} of x% PCL-y% ACPCL films were an average of 57.3 7.2% in comparison to 72.0-17.3% for the 100% PCL-dimethacrylate control (Table 3). Prior to cross linking, the Tm of all materials besides 85% PCL-15% ACPCL were great than 37° C. (Table 2). Crosslinking of the materials resulted in a Tₘ reduction to 43.4-29.7° C. for y=4.16-14.50% copolymer films (Table 3) due to the restricted mobility of the crosslinked polymer chains. This reduced chain mobility also disrupted the alignment of chains after melting, as indicated by a reduction in the percent crystallinity (X_{c}) after crosslinking. There was a dependence of the thermal properties, except for T_{g}, on molar composition for the crosslinked polymers (FIG. 3), as amorphous ACPCL disrupted the crystallinity of PCL and lowered the Tₘ, X_{c}, crystallization temperature (T_{c}), and enthalpy of crystallization (AHC). The X_{c} generated was similar to branched PCL crosslinked films, indicating that switch-like shape recovery is possible with these SMPs. Crosslinking produced a library of SMPs with switching temperatures (i.e., Tₘs) near 37° C. and sufficient X_{c} for complete shape recovery and switch-like behavior in physiological applications.

TABLE 2
Gel content and thermal properties of crosslinked x % PCL-y % ACPCL SMP films

| | X_{G} | Tₘ | ΔHₘ | X_{c} | T_{c} | ΔH_{c} | T_{g} |
|---|---|---|---|---|---|---|---|
| Composition | [%]^{a)} | [° C.] | [J/g] | [%]^{b)} | [° C.] | [J/g] | [° C.] |
| 100% PCL-dimethacrylate | 72.0 ± 17.3 | 48.1 ± 0.4 | 48.2 ± 0.5 | 34.6 ± 0.4 | 19.5 ± 1.0 | 48.6 ± 0.4 | −54.2 ± 3.0 |
| 96% PCL-04% ACPCL | 63.0 ± 8.6 | 43.4 ± 1.2 | 44.6 ± 3.2 | 32.0 ± 2.3 | 15.8 ± 0.9 | 43.2 ± 6.1 | −56.9 ± 0.1 |
| 94% PCL-06% ACPCL | 60.3 ± 21.3 | 37.9 ± 0.9 | 39.1 ± 5.3 | 28.0 ± 3.8 | 2.4 ± 0.5 | 38.7 ± 4.8 | −58.8 ± 4.9 |
| 89% PCL-11% ACPCL | 49.0 ± 6.2 | 37.9 ± 0.7 | 38.7 ± 1.6 | 27.7 ± 1.2 | −2.1 ± 0.7 | 36.5 ± 0.8 | −57.1 ± 1.5 |
| 88% PCL-12% ACPCL | 64.1 ± 3.1 | 33.4 ± 1.2 | 33.7 ± 1.1 | 24.2 ± 0.8 | −8.7 ± 0.2 | 31.4 ± 2.2 | −58.7 ± 2.2 |
| 85% PCL-15% ACPCL | 50.3 ± 0.6 | 29.7 ± 0.2 | 28.3 ± 2.7 | 20.3 ± 1.9 | −13.9 ± 0.8 | 17.2 ± 0.9 | −57.5 ± 1.1 |

^{a)}X_{G} = m_{extracted}/mᵢₙᵢₜᵢₐₗ × 100%, where m_{extracted} is the mass after incubating in dichloromethane for 2 days and subsequently drying, while mᵢₙᵢₜᵢₐₗ is the initial mass;
^{b)}X_{C} = ΔHₘ/ΔHₘ^{o} × 100%, where ΔHₘ^{σ} = 139.5 J/g, the enthalpy of fusion for 100% crystalline PCL.

Mechanical properties of the SMP test films were assessed isothermally at 37° C. to determine suitability for vascular applications. The elasticity was of the same order of magnitude or one lower than the 100% PCL-dimethacrylate control (Table 4, for y=4.16-14.50%: tensile modulus at 37° C.). (Eₜₙ′(37° C.=55.0-2.2 MPa) that may be considered desirable compliance for vascular applications. The higher y% ACPCL crosslinked copolymer films displayed an order of magnitude lower Eₜₙ′(37° C.) that more closely matches that of native arteries and was primarily the result of these materials partially or fully melting at 37° C. Stress-to-break, σₘₐₓ, was between 3.3-0.12 MPa and most of the materials had good ductility at 37° C. with over 85% strain-to-break, εₘₐₓ, for every test film but 85% PCL-15% ACPCL (εₘₐₓ=28%). These experiments demonstrate that the library of crosslinked SMPs has appropriate extensibility and compliance for vascular applications.

TABLE 4
Mechanical and shape memory properties of crosslinked SMP films

| | Eₜₙ, 37° C. | εₘₐₓ | σₘₐₓ | Rᵣ(1) | Rᵣ(N) | R_{f}(N) |
|---|---|---|---|---|---|---|
| Composition | [MPa]^{a)} | [%]^{a)} | [MPa]^{a)} | [%]^{b)} | [%]^{b)} | [%]^{c)} |
| 100% PCL-dimethacrylate | 53.8 ± 36.7 | 199.5 ± 71.2 | 4.68 ± 0.3 | 99.7 ± 0.1 | 99.5 ± 1.4 | 98.3 ± 1.5 |
| 96% PCL-04% ACPCL | 55.0 ± 17.1 | 93.4 ± 135.5 | 3.3 ± 0.4 | 99.4 ± 0.8 | 99.4 ± 1.3 | 94.2 ± 1.2 |
| 94% PCL-06% ACPCL | 3.05 ± 2.6 | 253.0 ± 19.4 | 2.36 ± 0.9 | 93.7 ± 0.9 | 98.5 ± 0.6 | 98.7 ± 0.3 |
| 89% PCL-11% ACPCL | 4.53 ± 3.4 | 131.4 ± 81.9 | 0.77 ± 0.6 | 97.4 ± 0.7 | 99.7 ± 0.7 | 99.8 ± 0.2 |
| 88% PCL-12% ACPCL | 4.24 ± 1.1 | 84.5 ± 89.1 | 0.99 ± 0.6 | 99.9 ± 9.2 | 99.0 ± 0.2 | 98.8 ± 0.9 |
| 85% PCL-15% ACPCL | 2.18 ± 0.1 | 28.1 ± 32.2 | 0.12 ± 0.1 | 60.1 ± 0.6 | 86.9 ± 4.7 | 99.6 ± 0.2 |

^{a)}Mechanical properties determined by a tensile test with a stress ramp of 0.1 MPa min⁻¹ at 37° C.;
^{b)}Shape memory properties determined by stress - controlled thermomechanical cycling;
${R}_{r} ( N ) = \frac{{ε}_{1} ( N ) - {ε}_{p} ( N )}{{ε}_{1} ( N ) - {ε}_{p} ( N - 1 )}$ describes how well shape is recovered (εₚ(N)) in comparison to the beginning of the N^{th} cycle (εₚ(N − 1) after deforming to maximum strain ε₁ (N);
^{c)} ${R}_{f} ( N ) = \frac{{ε}_{u} ( N )}{{ε}_{1} ( N )}$ defines the ability to maintain programmed shape εₚ(N) after unloading of stress to yield the temporary shape εᵤ(N).;
A 96% PCL-04% ACPL test film with X_{G} = 36.7 ± 8.6% had R_{f}(1) = 99.9 ± 0.2, Rᵣ(N) = 99.8 ± 0.4%, and RAN) = 99.8 ± 0.1%.

### Example 3

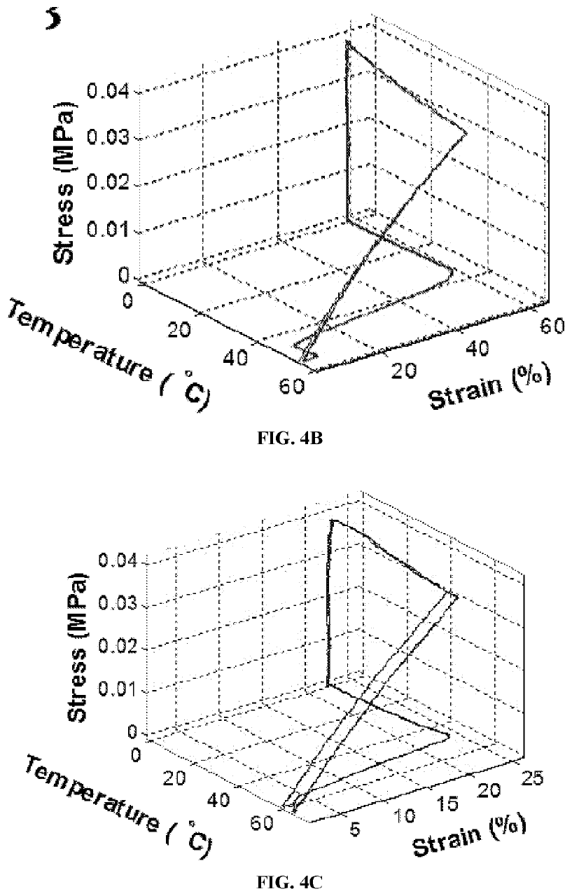

This Example describes the preparation of SMP shapes to evaluate shape memory properties by stress controlled thermomechanical cycling (FIGS. 4A to 4C). Closed-end polymer tubes (˜1.0-2.0 cm length, −0.90 mm in I.D., ˜1.0-1.6 mm O.D.) were prepared by dipping a poly vinyl alcohol (PVA)-coated 0.90 mm O.D. glass capillary in the polymer film preparatory solution and UV-crosslinking as above. Capillaries containing the tubes were dried and immersed in deionized H₂O and 100% ethanol before manually pulling the tubes off the capillaries. The tubes were washed with H₂O, dried, and the open side of the tube was closed by dipping it in polymer solution and UV crosslinking. A guitar shape comprised of 94% PCL-06% ACPCL was prepared by first laser etching (Epilog Laser, Golden, Colo.) a 2 mm PDMS mold containing a CAD-designed guitar, then pouring the 94% PCL-06% ACPCL polymer solution into the mold and UV crosslinking (365 nm, 26.1 J cm⁻², 290 mW cm⁻²) on a 48° C. hotplate.

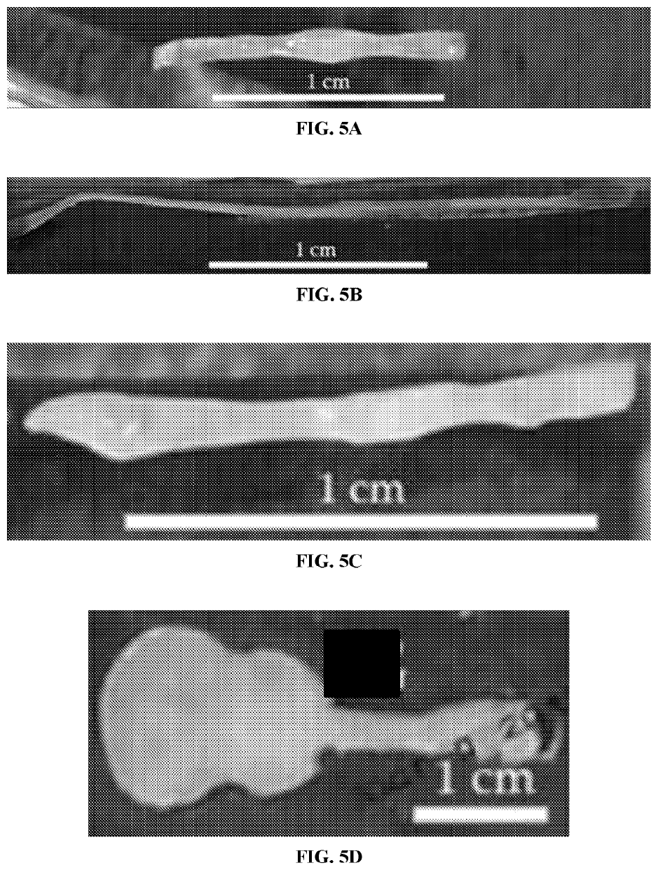

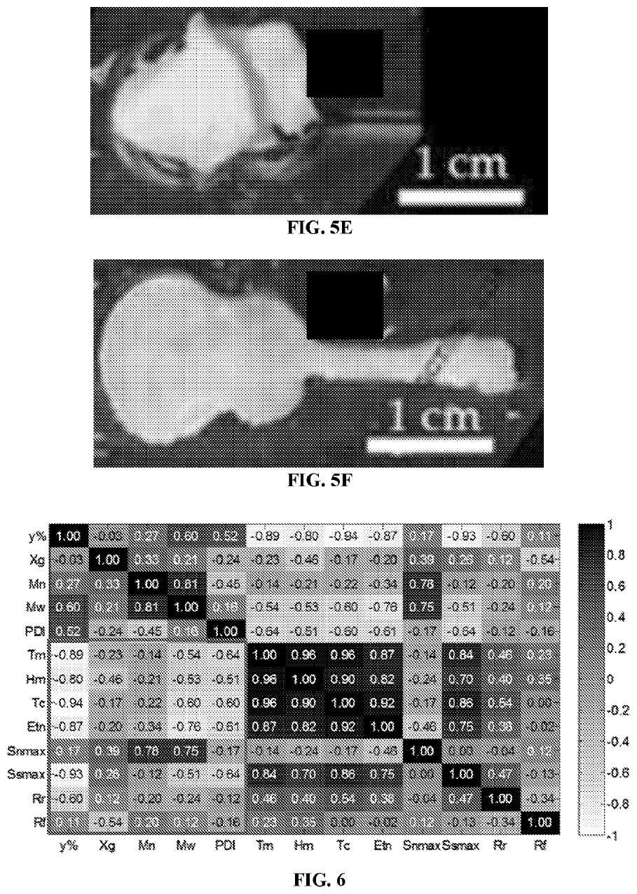

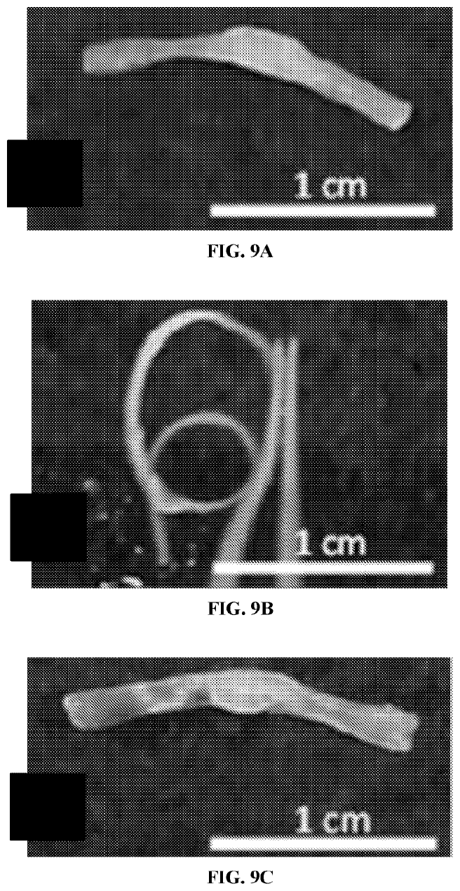

Shape recovery after the first cycle, Rᵣ(N), which indicated the quantitative ability of materials to recover their original shape (e.g., tubular shape), was over 98% for test films of every material composition except for 85% PCL 15% ACPCL (Rᵣ(N)=86.9+4.7%) (Table 4). Shape fixity (Rf) represents the ability of materials to be fixed in a temporary shape (e.g., thread-like shape) and was over 98% for select films of every material composition (Table 4). Depiction of three consecutive thermomechanical cycles for 96% PCL-04% ACPCL and 89% PCL-11% ACPCL (FIGS. 4B and 4C) illustrated the repeatable nature of shape programming and recovery for these SMPs. Shape memory demonstrations further affirmed the utility of the materials in biomedical applications (FIGS. 5A to 5F and FIGS. 9A to 9C), including the desired thread-to-tube transition for minimally-invasive catheter or laparoscope deployment in arterial bypass grafting at 37° C. Most copolymers possessed exceptional, tightly-controllable shape memory capabilities.

### Example 4

This Example evaluated structure-function relationships to better elucidate correlations of material properties (Tₘ, ΔHₘ, T_{c}, Eₜₙ′(37° C.), σₘₐₓ, εₘₐₓ, Rᵣ(N), R_{f}(N)) with physicochemical properties (y% ACPCL, Mₙ, M_{w}, PDI, X_{G}). Briefly, a 13×10 matrix was constructed containing the mean values of each variable to be compared (13 variables) for each of the 10 polymer films (FIG. 6). Matrix values were standardized to their Z-score for more apt comparison between variables, and a covariance matrix was computed and plotted using MATLAB (Math Works Inc., Natick, Mass.).

Covariances (covs) closest to the absolute value of 1 indicate the strongest correlations between variables, with positive and negative values indicating direct and inverse relations, respectively. Thermal properties, Eₜₙ′(37° C.), and σmax correlate strongly with y% ACPCL (cov=−0.80-0.94), indicating a dominant role of molar composition on these properties. Without being bound by theory or mechanism, this dominance of molar composition on certain material properties can be explained by the fact that altering allyl content simultaneously changes both the crystallinity and spacing of netpoints of the crosslinked networks. Rᵣ(N) was also impacted by molar composition (cov=−0.60), although it is conceivable that programming parameters (e.g., fixation and deformation temperature, stress or strain rate) could be adjusted to improve Rᵣ(N) for higher y% ACPCL copolymers. Mₙ correlated strongly with εₘₐₓ (cov=0.78), indicating that Mₙ may be increased to improve the extensibility of these SMPs. Further, X_{G} can be adjusted to increase R_{f}(N) (cov=−0.54) and AHm (cov=−0.46). Thus, several material properties are affected by molar composition, and many can be tuned via modulation of other physicochemical properties to comprise PCL-ACPCL SMPs with certain thermal, mechanical, and shape memory properties.

### Example 5

This Example describes vascular compatibility studies utilized to assess the biocompatibility of the films. Human umbilical vein endothelial cells (HUVECs) were seeded on polymer films and their viability was measured over the course of four days using the resazurin assay (FIG. 7). To prevent cell attachment on tissue culture polystyrene (TCPS) underneath test films, wells were coated with 1% agarose solution. Agarose-coated wells were dried, washed with 100% ethanol, UV sterilized, and washed with MesoEndo Endothelial Cell Growth Media (Cell Applications, Inc. San Diego, Calif.). Ethanol-leached, media soaked polymer disks (˜31 mm², −50 um thick) were then placed on the agarose-coated wells, and Passage 5 red fluorescent protein-expressing HUVECs (P5 RFP-HUVECs) (470 cells mm²) were seeded directly on the film surfaces, TCPS (positive control), and 1% agarose (negative control). After 1.5 hours, 150 μL of media was added.

Viability was assessed at 9-, 35-, and 91-hour time points via the resazurin assay. Briefly, resazurin (5 μM in MesoEndo) was added to each well, incubated for 4 hours at 37° C. and 560/590 nm excitation/emission of the supernatant was read on an Infinite® M1000 Pro plate reader (Tecan Group Ltd, San Jose, Calif.). Viable cell number was calculated based on a standard curve of RFP-HUVEC fluorescence on TCPS, and % cell viability was normalized to TCPS controls. All samples were tested in biological quadruplicates.

100% PCL (Sigma-Aldrich, Mₙ=70-90 kDa) is known to be biocompatible and was therefore selected as a control film. Nine hours post-seeding, there was no statistically significant difference in HUVEC viability on test SMP films (60.0-65.2% relative to TCPS) compared to 100% PCL (59.4±4.9%). At later timepoints, HUVEC viability on all copolymer films (102.9-106.7% for 35 hours and 85.0-103.0% for 91 hours) was greater than that on 100% PCL (66.0±14.4% and 64.1±32.0%, respectively).

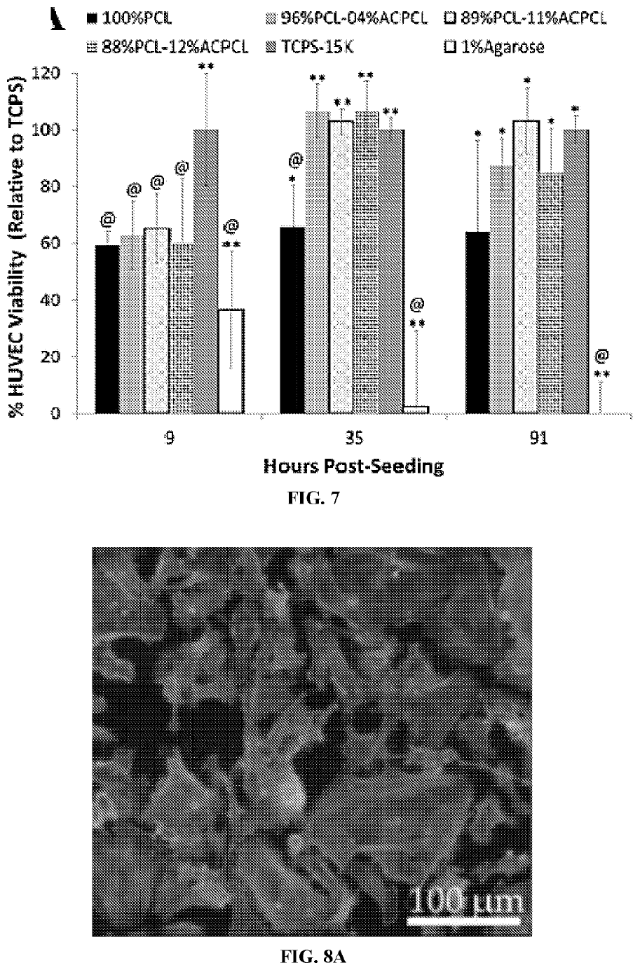

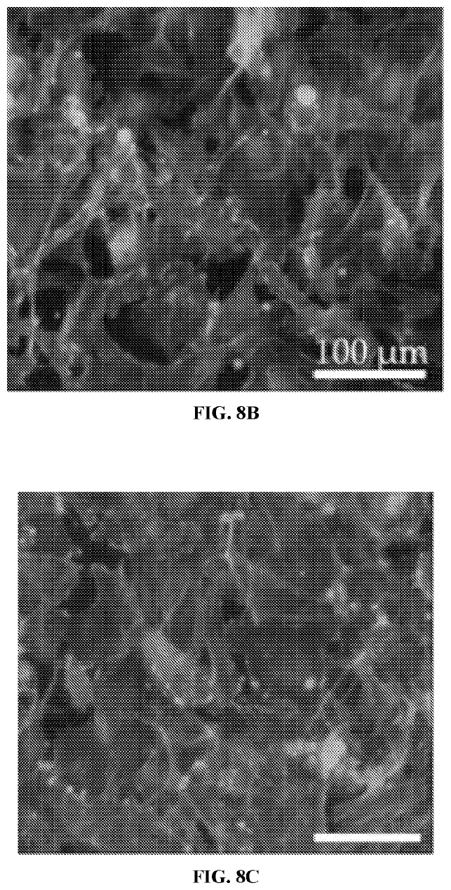

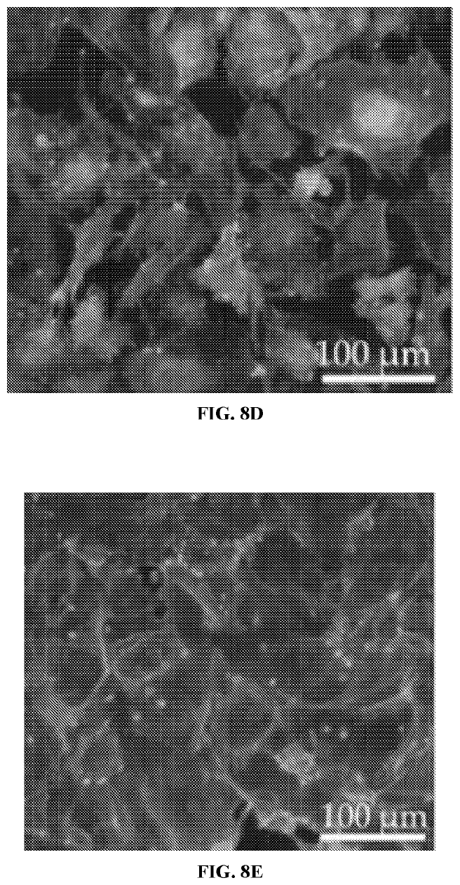

Additionally, cell morphology was evaluated by seeding P5 human coronary artery endothelial cells (hCAECs) (Cell Applications, Inc. San Diego, Calif.) directly onto polymer disks. After 3 days of incubation on the disks or TCPS controls, cells were fixed with 4% paraformaldehyde (15 minutes), permeabilized with 0.5 Triton X-100 (10 min), and blocked with 10% Bovine Serum Albumin (30 min). Cells were then incubated with 2 UM Ethidium Homodimer-1 (10 min) and 50 μM Alexa Fluor® 488 Phalloidin (Molecular Probes, Eugene, Oregon.) (20 min). Cells on polymer surfaces were imaged on a LSM 510 META Inverted Confocal Microscope (Carl Zeiss, LLC, Thormwood, N.Y.), while TCPS controls were imaged with a Nikon Eclipse Ti inverted fluorescence microscope (Nikon Instruments Inc., Melville, N. Y.). Images were post-processed and analyzed using ImageJ software (NIH, Bethesda, Md.). Confocal microscopy of hCAECs on all films after 3 days demonstrated trademark cobblestone morphology (FIGS. 8A to 8E). Thus, the SMPs were compatible with vascular ECs and could potentially endothelialize when used as an arterial bypass graft.

### Example 6

This Example describes an in vivo arterial bypass grafting procedure conducted in order to assess the therapeutic viability of the present compounds and grafts. A SMP tubular graft was utilized to provide a conduit for blood flow past an occluded region in a model of rat carotid artery ligation in vivo. The 89% PCL-11% ACPCL copolymer was chosen as the tubular construct because it possessed shape memory properties (R, and R, >99%), a Tₘ close to body temperature (37.9° C.), and high EC biocompatibility after 91 hours (103.0%) (FIGS. 9A to 9C).

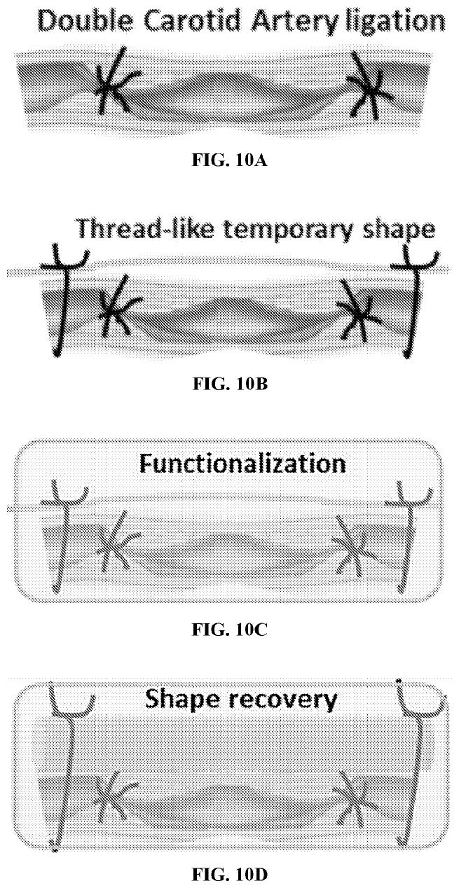

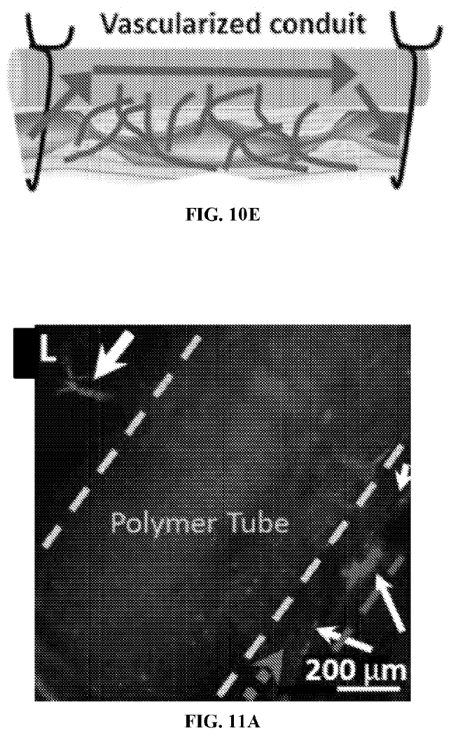

Immediately prior to surgery, closed-end SMP grafts (0.9 cm I.D., 1.2 cm O.D., 1.5 cm length) comprised of 89% PCL-11% ACPCL were UV sterilized and collagen gels containing C16 and Ac-SDKP were prepared. Sprague Dawley rats were subjected to a double ligature of the left common carotid artery as a model of complete blood cessation (FIG. 10A). Test groups included “Polymer+Peptide”, “Peptide Only”, and “Untreated” test groups. In the “Polymer+Peptide” group, SMP tubes with tow closed ends were placed over the entire occluded area immediately following the ligations, each tube end was tied to the native artery by suturing, and the construct and artery were embedded in the collagen gel containing pro-angiogenic C16 and anti-inflammatory AC-SDKP peptides by cotton swab application (FIGS. 10A to 10E). In the “Peptide Only” group, only the peptide-containing collagen gel was applied immediately following the ligations. No polymer or peptides were applied in the “Untreated” group. All incisions were sutured closed using non-degradable sutures. Rats were given buprenorphine 0.05 mg/kg SQ every 8-12 hours as needed for pain and monitored for two weeks.

Following the two-week implantation, fluorescence microangiography was performed using 0.1 um diameter FluoSpheres® Carboxylate-Modified Red Fluorescent Microspheres (Life Technologies Corp., Carlsbad, Calif.) in heparinized saline (1:20 dilution) to assess areas of capillary growth and blood perfusion. Within 3 hours of the perfusion event, the beads were observed using a LSM 510 META Inverted Confocal Microscope (Carl Zeiss, LLC, Thormwood, N.Y.). Rat tissue around the polymer-artery interface was embedded in optical cutting temperature (OCT), frozen at −80° C. for 24 hours, and sectioned (5 μm sections) using a cryotome.

To identify vascular cells around the polymer artery interface, frozen sections were stained with mouse anti-rat phycoerythrin (PE)-conjugated CD31 antibodies (clone TLD-3A12, BD Biosciences) as an endothelial and leukocyte cell marker, then counter-stained with Hoechst 33258 nuclear stain (Life Technologies, Inc.). The Nikon Eclipse Ti inverted fluorescence microscope (Nikon Instruments Inc., Melville, N. Y.) was used to capture images of the IF-stained OCT sections.

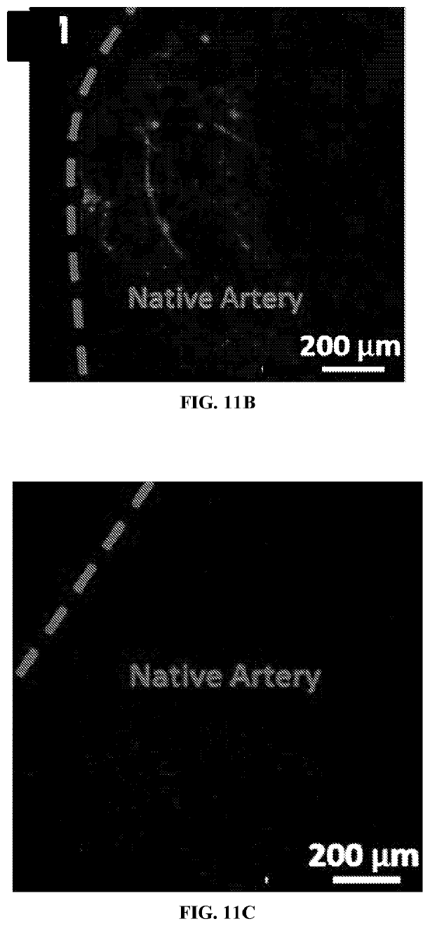

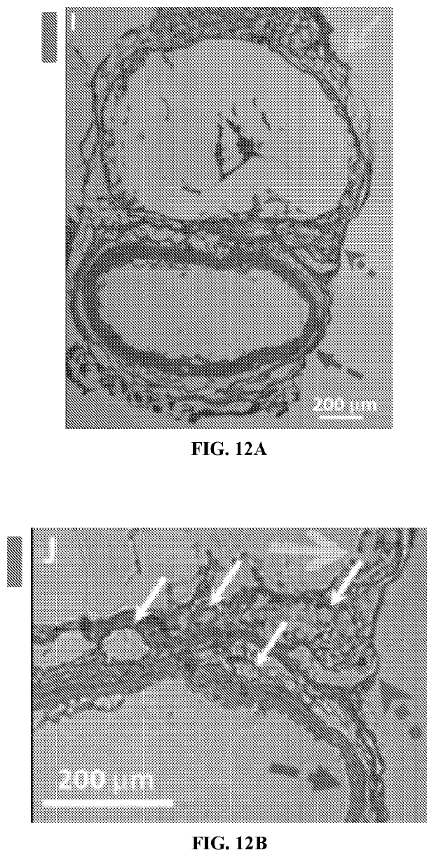

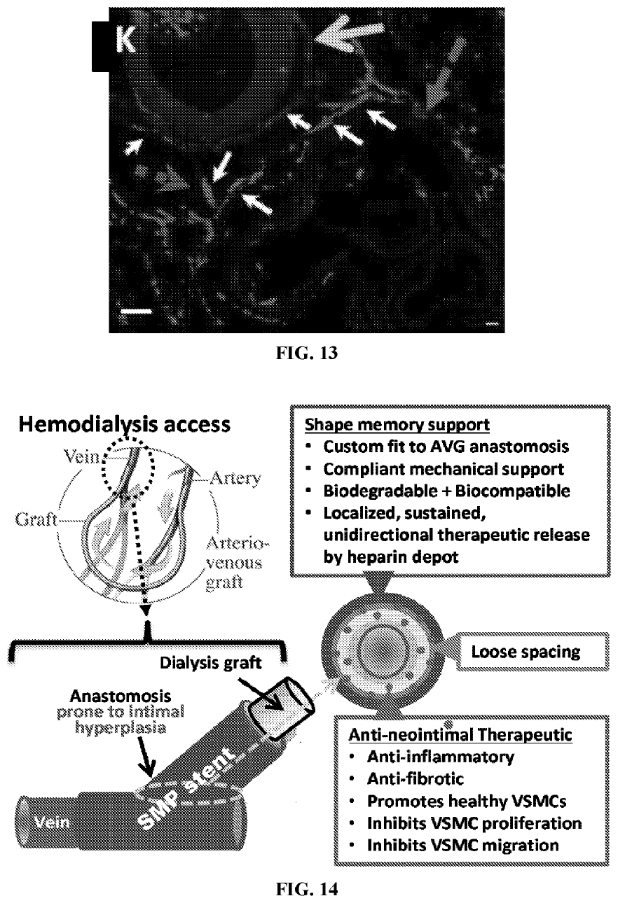

After 2 weeks, the very strong fluorescent signal in the “Polymer+Peptide” group from detection of fluorescent beads using fluorescence microangiography (FIG. 11A) indicating that blood was flowing through the tubular construct. There is little to no visible fluorescence in the other test groups (FIGS. 11B and 11C), signifying near-complete occlusion without this combination treatment. Observation of a purple/pink microvessel network from H & E staining (FIGS. 12A and 12B), and fluorescence of CD31+vascular cells (FIG. 13) for the “Polymer+Peptide” group illustrated anastomosis between the polymer tube and native artery via capillary interconnectivity.

### Example 7

This example demonstrates characteristics of embodiments of the present invention.

One embodiment of the present invention is a composition of crosslinked x% PCL-y% ACPCL between y=10-15% to enable custom fitting capabilities, regulation of healthy VSMC phenotypes and surface coating-mediated, sustained, unidirectional MK2i delivery.

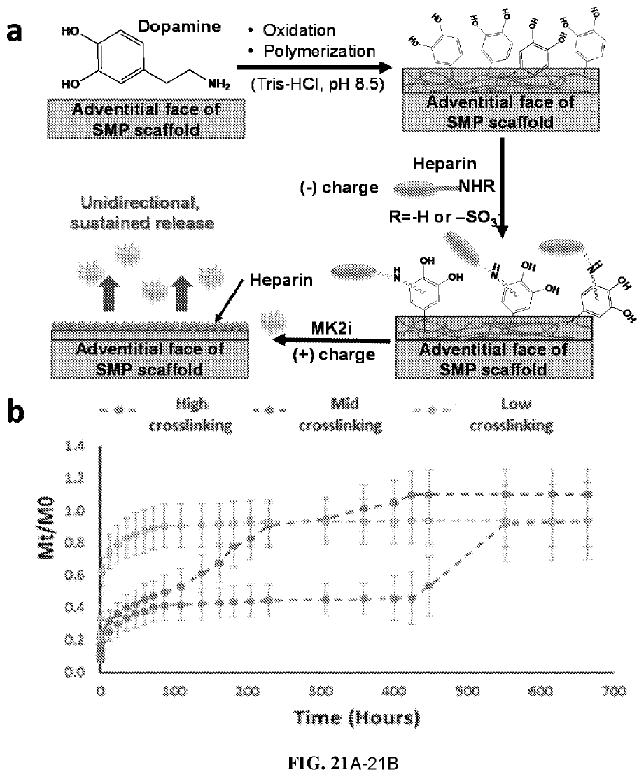

This embodiment includes SMPs that have at least one of, in any combination: high shape fixity and shape recovery (>95%) (see FIG. 15) to ensure efficient wrapping of the external support; melting temperatures <37° C. (FIG. 15) to enable shape molding around body temperature; tensile modulus at 37° C. Eₜₙ′(37° C.), of 1-100 MPa to provide mechanical support for healthy adaptation of the venous grafts in the arterial circulation while obviating any ill effects induced from compliance mismatches between the graft and vein; pores ˜750 um in diameter with high porosity (>50%) (FIG. 20) to foster neoadventitial growth and extension beyond the outside of the external stent for efficient nutrient and oxygen transport; slow degradation (at least several months) to maintain sufficient mechanical support during the pivotal adaptation period of the vein to the arterial circulation; heparin coating (“depot”) to enable unidirectional, sustained release of MK2i. The positively charged MK2i can be loaded into heparin-containing hydrogels in a manner similar to other heparin-binding peptides and released based on heparin concentration, and desirable MK2i release profiles (50 μg MK2i/day) to achieve 100 μM/day in a volume equivalent to a typical venous anastomosis over 28 days. Heparin concentration controls both the density of anionic charges and the porosity of the heparin layer to provide variable “windows for release” for drugs like cationic MK2i. MK2i concentrations can also be con trolled to alter release amounts and kinetics.

### Example 8

This example demonstrates the custom fitting process of the SMPs of the present invention. The present invention has excellent shape memory capabilities at body temperature. SMP external stent examples of the present invention may be comprised of 5 different x% PCL-v% ACPCL copolymers (y=10, 11, 12, 13, 14 and 15% with melting temperatures between 28-37° C.). To start, stents may be made 8 mm in diameter to be loose-fitting around typical human saphenous veins (HSVs) (˜2 mm space to allow neoadventitial growth), 0.5 mm in thickness to allow for significant deformability, with a 3.1 cm long arm and 1 cm side arm to sufficiently cover the venous anastomosis. Similar to other external meshes, macropores 750 μm in diameter with >50% porosity may be fabricated to prevent ischemia and promote adventitial growth and outward remodeling.

The invention includes SMP external meshes with melting temperature that fall within vascular access operating temperatures (28-37° C.) and contain macropores: A positive mold may be 3D printed (FIG. 20A-20B) and assembled, then embedded with polydimethylsiloxane (PDMS) to make a negative mold containing channels (pore generators) (FIG. 20C-20D). The PDMS mold may then be placed in the glass mold (FIG. 20D). The space between the PDMS and glass negative mold may then be filled with polymer solution [25 (wt/vol) % of x% PCL-y% ACPCL (y=10, 11, 12, 13, 14 and 15%), 1 (wt/vol) % 2,2-dimethoxy 2-phenylacetophenone in dichloromethanel and UV cross linked (4.89 J/cm², 18.1 mW/cm²) 4. The PDMS is then mechanically cut to retain the SMP stent (FIG. 20E). These molds may be adjusted to the y shape format (FIG. 20F).

A heparin coating may be achieved by first forming a thin poly (3,4-dihydroxy-L-phenylalanine) (poly(DOPA)) layer on the luminal face of the SMP. Then the amine group of heparin may be covalently conjugated to poly(DOPA) (FIG. 21A). The luminal face of SMP supports may be immersed in a mixture of Iris (pH 8.5) and ethanol (Vₜᵣᵢₛ:Vₑₜₕₐₙₒₗ=7:3) with L-DOPA for 12 hours. The DOPA-coated face may then be immersed in heparin solutions (pH 7.4) with variable concentrations (1, 10, and 50 g/L) for 24 hours. AlexaFluor568-conjugated MK2i (10, 100, or 1000 μM in 100 μL PBS) may then be incubated with the heparin-coated stent samples for 2 hours at 37° C. Fresh PBS is then added at each timepoint (0.25, 0.5, 1, 2, 4, 8, 12, and 24 hours, then daily for 28 days) to mimic the in vivo “infinite sink” condition as we have previously shown. Collected supernatants may be read on a plate reader (excitation/emission of 578/603 nm) and compared to unloaded SMP/heparin and drug-AlexaFluor568 alone controls to derive a standard curve.

MK2i doses in this range should allow 50 μg/day of MK2i to be released over 28 days to achieve 100 μM/day, the effective dose used to prevent vein graft intimal hyperplasia in a volume equivalent to a typical antecubital vein (3 mm diameter, 230 μm thickness). While vein wall thickening continues over 12 weeks into arterial exposure, a 2-4 week sustained release profile of MK2i may be ideal because the majority of VSMC proliferation and migration occurs within this window, and MK2i inhibits the VSMC actions by its anti-hyperplasia effects.

As expected (FIG. 21B), the most integrated depot layer (highest crosslinking with smallest mesh size) yields the most sustained release, whereas the least integrated scaffolds (largest mesh size) exhibits the most burst release. This data indicates that the integrity of the depot layer can be altered to achieve this sustained release profile over the critical 2-4 week time period when neointimal formation is most accelerated owing to VSMC proliferation and migration. However, as accelerated degradation of the used depot material (gelatin gel) is expected in vivo, anionic heparin coatings may be used instead to load the cationic MK2i.

Without being bound by theory or mechanism, capillary formation arose from the pro-angiogenic, anti-inflammatory activities of C16 and AC-SDKP peptides distributed throughout the polymer-artery interface, providing a means for blood to be diverted into the polymer construct and return to the native artery via a pressure gradient generated following the direction of blood cessation. Thus, the tubular construct attached with the native vasculature via capillary connection can provide an additional conduit with the occluded artery, and can eliminate the need to perform transection of an artery during arterial bypass grafting procedures.

### OVERVIEW OF TERMS

It will be understood that various details of the presently disclosed subject matter can be changed without departing from the scope of the subject matter disclosed herein. Furthermore, the description provided herein is for the purpose of illustration only, and not for the purpose of limitation.

While the terms used herein are believed to be well understood by one of ordinary skill in the art, the definitions set forth herein are provided to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

The terms “comprising,” “including,” and “having” are intended to be inclusive and mean that there may be additional elements other than the listed elements.

Following long-standing patent law convention, the terms “a,” “an,” and “the” refer to “one or more” when used in this application, including the claims. Thus, for example, reference to “a polymer” includes a plurality of such polymers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments±50%, in some embodiments±40%, in some embodiments±30%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Throughout this document, references are mentioned. All such references are incorporated herein by reference.

### REFERENCES

1. A. Lendlein, S. Kelch, Angewandte Chemie International Edition 2002, 41, 2034.
2. W. Small, P. Singhal, T. S. Wilson, D. J. Mait-land, Journal of materials chemistry 2010, 20, 3356.
3. A. Lendlein, M. Behl, B. Hiebl, C. Wischke, Expert review of medical devices 2010, 7, 357.
4. M. C. Serrano, G. A. Ameer, Macromolecular bioscience 2012, 12, 1171.
5. C. Liu, H. Qin, P. T. Mather, Journal of materials chemistry 2007, 17, 1558.
6. A. Lendlein, S. Kelch, Angew Chem Int Ed Engl 2002, 41, 2035.
7. M. Behl, A. Lendlein, Materials Today 2007, 10, 20.
8. J. Leng, X. Lan, Y. Liu, S. Du, Progress in Materials Science 2011, 56, 1077.
9. I. A. Rousseau, Polymer Engineering & Science 2008, 48, 2075.
10. A. Lendlein, R. Langer, Science 2002, 296, 1673.
11. M. Ebara, K. Uto, N. Idota, J. M. Hoffman, T. Aoyagi, Advanced Materials 2012, 24, 273.
12. A. Garle, S. Kong, U. Ojha, B. M. Budhlall, ACS applied materials & interfaces 2012.
13. M. Uygun, M. A. Tasdelen, Y. Yagci, Macromolecular Chemistry and Physics 2010, 211, 103.
14. X. Xu, K. A. Davis, P. Yang, X. Gu, J. H. Henderson, P. T. Mather, Macromolecular Symposia 2011, 309-310, 162.
15. X. Wang, T. C. Boire, C. Bronikowski, A. L. Zachman, S. W. Crowder, H.-J. Sung, Tissue Engineering Part B: Reviews 2012, 18, 396.
16. M. A. Woodruff, D. W. Hutmacher, Progress in Polymer Science 2010, 35, 1217.
17. H. Jeong, B. Kim, Y. Choi, Polymer 2000, 41, 1849.
18. M. A. Woodruff, D. W. Hutmacher, Progress in Polymer Science 2010, 35, 1217.
19. D. L. Safranski, K. E. Smith, K. Gall, Polymer Reviews 2013, 53, 76.
20. G. Mani, M. D. Feldman, D. Patel, C. Agrawal, Biomaterials 2007, 28, 1689.
21. S. J. Head, J. Borgermann, R. L. J. Osnabrugge, T. M. Kieser, V. Falk, D. P. Taggart, J. D. Puskas, J. F. Gummert, A. P. Kappetein, European heart journal 2013, 34, 2873.
22. A. L. Zachman, S. W. Crowder, O. Ortiz, K. J. Zienkiewicz, C. M. Bronikowski, S. S. Yu, T. D. Giorgio, S. A. Guelcher, J. Kohn, H.-J. Sung, Tissue Engineering Part A 2012, 19, 437.
23. X. Hu, X. Chen, S. Liu, Q. Shi, X. Jing, Journal of Polymer Science Part A: Polymer Chemistry 2008, 46, 1852.
24. A. Mahmud, X.-B. Xiong, A. Lavasanifar, Macromolecules 2006, 39, 9419.
25. A. S. Karikari, W. F. Edwards, J. B. Mecham, T. E. Long, Biomacromolecules 2005, 6, 2866.
26. A. Lendlein, A. M. Schmidt, M. Schroeter, R. Langer, Journal of Polymer Science Part A:Polymer Chemistry 2005, 43, 1369.
27. C. G. Pitt, F. I. Chasalow, Y. M. Hibionada, D. M. Klimas, A. Schindler, Journal of applied polymer science 1981, 26, 3779.
28. C. M. Yakacki, R. Shandas, C. Lanning, B. Rech, A. Eckstein, K. Gall, Biomaterials 2007, 28, 2255.
29. L. Xue, S. Dai, Z. Li, Macromolecules 2009, 964.
30. G. Zhu, G. Liang, Q. Xu, Q. Yu, Journal of applied polymer science 2003, 90, 1589.
31. F. Li, W. Zhu, X. Zhang, C. Zhao, M. Xu, Journal of applied polymer science 1999, 71, 1063.
32. B. Guo, Y. Chen, Y. Lei, L. Zhang, W. Y. Zhou, A. B. Rabie, J. Zhao, Biomacromolecules 2011, 12, 1312.
33. D. M. Feldkamp, I. A. Rousseau, Macromol Mater Eng 2010, 295, 726.
34. A. W. McClung, G. Tandon, J. Baur, Mech Time-Depend Mater 2013, 17, 39.
35. K. Gall, C. M. Yakacki, Y. Liu, R. Shandas, N. Willett, K. S. Anseth, Journal of Biomedical Materials Research Part A 2005, 73A, 339.
36. M. V. Lancaster, U.S. Pat. No. 5, 501, 959, 1996.
37. M. C. Serrano, R. Pagani, M. Vallet-Regi, J. Peña, A. Rámila, I. Izquierdo, M. T. Portolés, Biomaterials 2004, 25, 5603.
38. C. X. F. Lam, D. W. Hutmacher, J.-T. Schantz, M. A. Woodruff, S. H. Teoh, Journal of Biomedical Materials Research Part A 2009, 90A, 906.
39. W. P. Williamson IV, P. A. Spence, G. A. Keller, C. R. Robinson, T. J. Ward, U.S. Pat. No. 7,722,642,2010.
40. M. Song, H. Jang, J. Lee, J. H. Kim, S. H. Kim, K. Sun, Y. Park, Biomaterials 2014, 35, 2436.

We claim:

1. A method for treating a vascular condition in a patient, using a shape memory polymer vascular graft having a shape transition temperature, the method comprising:
forming a vascular graft in an original shape;
administering the vascular graft to an treatment site within a vasculature of the patient, while the vascular graft is in the original shape;
elevating the temperature of the vascular graft above the shape transition temperature and transforming the vascular graft from the original shape to a transplanted shape;
wherein when the vascular graft is in the transplanted shape, the vascular graft conforms to one or more features of the vasculature of the patient at the treatment site.

2. The method of claim 1, wherein administering the vascular graft to an treatment site comprises advancing the vascular graft in the original shape through the vasculature of the patient, via a catheter.

3. The method of claim 1, wherein administering the vascular graft to the treatment site comprises advancing the vascular graft alongside a treatment site of the patient using a tunneling device.

4. The method of claim 1, wherein administering the vascular graft to the treatment site comprises coupling the vascular graft to the treatment site by one or more sutures.

5. The method of claim 1, wherein administering the vascular graft omits the use of any sutures, and wherein the vascular graft is attached to the vasculature of the patient by the transformation of the vascular graft from the original shape to a transplanted shape

6. The method of claim 1, further comprising administering one or more bioactive agents that facilitate angiogenesis in advance of, along with, or after administering the vascular graft to the treatment site.

7. The method of claim 1, wherein the vascular graft is biodegradable and degrades within the patient such that the vascular graft does not need to be removed from the patient.

8. The method of claim 1, wherein the shape transition temperature is a temperature ranging from 20° C. to 50° C.

9. The method of claim 8, wherein the shape transition temperature is a temperature ranging from 28° C. to 37° C.

10. The method of claim 8, wherein exposure to body heat of the patient elevates the temperature of the vascular graft above the shape transition temperature without the use of an external heat source.

11. The method of claim 1 wherein the shape memory polymer of the vascular graft is a copolymer of a first monomer and a second monomer, wherein the first monomer is allyl-functionalized and cross-linkable monomer, and the second monomer is not cross-linkable.

12. The method of claim 11, wherein the first monomer is allyl carboxylate polycaprolactone and the second monomer is polycaprolactone.

13. The method of claim 12, wherein the first monomer is present in an amount ranging from greater than 0 mol % to 15 mol %.

14. The method of claim 1, wherein the original shape of the vascular graft is a thread, a sheet, tubular shape, a shape corresponding to a treatment site, a vascular patch, a vascular bypass graft, a vascular stent, or a combination thereof.

15. The method of claim 1, wherein the method is a bypass graft and the the transplanted shape of the vascular graft is a sutureless sheath configured to surround a native artery and a bypass vessel.

16. The method of claim 1, wherein when the shape memory polymer of the vascular graft is at a temperature of 37° C., a tensile modulus of the vascular graft is about equal to a tensile modulus of a native artery.

17. A vascular bypass method using an allyl carboxylate polycaprolactone-polycaprolactone copolymer vascular graft, the method comprising:
coupling a bypass vessel with an arterial vessel to form a bypass junction between the bypass vessel and the arterial vessel;
placing the vascular graft, in the form of a sheet, stent, or sleeve, at the bypass junction such that the vascular graft surrounds the bypass junction, while the vascular graft is at a temperature below a shape transition temperature of the allyl carboxylate polycaprolactone-polycaprolactone copolymer; and
elevating the temperature of the vascular graft above the shape transition temperature to cause the vascular graft to conform to the shape of the bypass junction and at least a portion of each of the bypass vessel and the arterial vessel,
wherein the bypass vessel is sealed to and secured against the arterial vessel by the vascular graft when the vascular graft is conformed to the shape of the bypass junction,
wherein the shape transition temperature is within 5° C. of a body temperature of a patient.

18. The vascular bypass method of claim 17, wherein the bypass vessel is a veinous vessel.

19. The vascular bypass method of claim 17, wherein the bypass vessel is a synthetic material.

20. A method for treating a vascular condition in a patient, using a shape memory polymer vascular graft having a shape transition temperature, the method comprising:
forming a vascular graft in an original shape;
applying a stress to the vascular graft to deform the graft from the original shape to a temporary shape;
reducing the temperature of the vascular graft below the shape transition temperature to retain the vascular graft in the temporary shape;
administering the vascular graft to a treatment site within a vasculature of the patient;
elevating the temperature of the vascular graft above the shape transition temperature to transform the vascular graft from the temporary shape to a transplanted shape;
wherein when the vascular graft is in the transplanted shape, the vascular graft conforms to one or more features of the patient's vasculature at the treatment site.
